SEQUENCE LISTING (9 sequences)

SEQ ID NO: 1 - DNA, 2279 bp, Human
ggcacgagcgaagatggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatgaaacccgtatagttcacaatagagctcagggagcccctaactcttccaaaccacatgggagacagtttccttcatgcccaagcctgagctcagatccagcttgcaactaatccttctatcatctaacatgcactacttggaaagatctaagatctgaatcttatcctttgccatcttctgttaccatatggtgttgaatgcaagtttaattaccatggagattgttttacaaacttttgatgtggtcaagttcagttttagaaaagggagtctgttccagatcagggccagaactgtgcccaggcccaaaggagacaactaactaaagtagtgagatagattctaagggcaaacatttttccaagtcttgccatatttcaagcaaagaggtgcccaggcctgaggtactcacataaatgctttgttttgctggtgatttaaccagtgcttggaaaaatcttgcttggctatttctgcatcatttcttaaggctgccttcctctctgagtacgttgccctctgtgctatcaatcatcttatcatcaattattagacaaatcccactggcctacagtcttgcttctgcagcacccactttgtctcctcaggtagtgatgaattagttgctgtcacaaaaggagggaagtagcacccaaattaaattgcttaagagaggaaatgtacatcttgtataacttagggagcgaagaaaatgtaggcgcgaaagtgaaaagtgaggcagctagttcttcctattccattctcgaccaacctgccctttcttaatatgactagtggtcttgatgctagagtcaacttactctgttgctggctttagcagagaataggaggaaccatatgaaaaagatcaggctttctgacttccatccccaaaacacatttaccagcatactccaaactgtttctgatgtgttccatgagaaaaggattgtttgctcaaaaagcttggaaaatactacacactccctttctccttctggagatcaacccacattagagtgtctaaggactcctgagaattcctgttacagtaaacaaaactaacgtaatctaccatttcctacactatttgagcatggaaatcatagtccccactctatgaaaacttaacgctttttggaagacatttctgtagcatgtcagtttggagaaatgatgagctacgccttgatgaaagaaccgtgttggtgctgctaagtttagccattatggtttttcctttctctctcttaagccttattcttcaactaaaagatgaggattaagagcaagaagttgggggggatgtgaaaataattttatgaggttgtctaaaatctcgtgcc

SEQ ID NO: 2 - protein, 306 aa, Human
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 3 - protein, 7 aa, Human
GCVKIKK

SEQ ID NO: 4 - protein, 8 aa, Human
IRYCWLRR

SEQ ID NO: 5 - protein, 9 aa, Human
VTTLCCGKN

SEQ ID NO: 6 - protein, 7 aa, Human
MLCCMRR

SEQ ID NO: 7 - protein, 8 aa, Human
MGCLGNSK

SEQ ID NO: 8 - protein, 8 aa, Human
MGCLGNSK

SEQ ID NO: 9 - protein, 10 aa, Human
GCMSCKCVLS

(19) United States
(12) Patent Application Publication
(10) Pub. No.: US 2003/0050236 A1
(43) Pub. Date: Mar. 13, 2003

(54) Compounds that enhance tumor death

(75) Inventors: Glyn Dawson, Chicago, IL (US); Seongeun Julia Cho, Hillsborough, NJ (US)

Correspondence Address: Gina N. Shishima, FULBRIGHT & JAWORSKI L.L.P., SUITE 2400, 600 CONGRESS AVENUE, AUSTIN, TX, 78701, US

(73) Assignee: The University of Chicago

(21) Appl. No.: 09/930,559

(22) Filed: Aug. 15, 2001

Related U.S. Application Data
(60) Provisional application No. 60/225,526, filed on Aug. 15, 2000.

(51) Int. Cl. A61K 38/17

(52) U.S. Cl. 514/12

(57) ABSTRACT

The present invention concerns compositions that modulate palmitoyl protein thioesterase 1 (PPT1) activity, as well as methods for using these compositions as a therapeutic treatment to inhibit a cancer cell, such as by promoting apoptosis of the cancer cell. It is contemplated that these compositions may be used in conjunction with other anti-cancer therapies such as chemotherapeutic agents. PPT1 modulators include polypeptide and peptides that competitively interact with PPT1, as well as PPT1 antisense and ribozyme constructs that prevent the expression of PPT1. Furthermore, the present invention also covers methods of screening for PPT1 modulators, as well as for levels of PPT1 amount or activity as a diagnostic tool.

This application claims priority to U.S. Provisional Patent Application serial No. 60/225,526 filed on Aug. 15, 2000, which is hereby incorporated by reference in its entirety.

The government may own rights in the present invention pursuant to grant number NS 36866 and HD 09402 from the National Institutes of Health.

### BACKGROUND OF THE INVENTION

1. Field of the Invention

The present invention relates generally to the fields of cancer therapy. More particularly, it concerns compositions and methods involving modulators of palmitoyl protein thioesterase 1 (PPT1) to induce apoptosis of cancer cells.

2. Description of Related Art

Approximately 1,220,100 new cases of cancer are expected to be diagnosed in the United States in 2000. In the same year, about 552,200 Americans are expected to die of cancer, which amounts to more than 1,500 deaths a day. Exceeded only by heart disease, cancer is the second leading cause of death in the United States, where 1 in 4 deaths is attributed to cancer. The National Institutes of Health (NIH) estimates overall annual costs for cancer to be approximately $107 billion, of which $37 billion is for direct medical costs (total of all health expenditures), $11 billion accounts for indirect morbidity costs (cost of lost productivity due to illness), and $59 billion related to indirect mortality costs (cost of lost productivity due to premature death). The incidence of cancer varies from country to country, yet it clearly poses an international health problem, for which no easy solutions have emerged.

A variety of treatment regimens have been developed to treat cancer (anti-cancer therapies), including surgery, chemotherapy, and radiation therapy. In some cases a combination of treatments is utilized. These treatments are employed with varying levels of efficacy as well as variable side effects. Side effects are related to damage caused to healthy tissue. Chemotherapy, for example, causes damage to cells in the bone marrow, gastrointestinal cells, cells involved in reproduction, and hair follicles, which in turn leads to side effects such as nausea, vomiting, hair loss, and fatigue. Compounds and compositions that can be used in conjunction with an anti-cancer therapy and that could either increase its efficacy or reduce the dosage, concentration, or number of administrations of that therapy would be beneficial patients in the treatment of cancer.

Palmitoyl protein thioesterase 1 (PPT1) was the first enzyme described which could remove palmitate from lipid-modified proteins on specific cysteine residues. Following its identification as the gene responsible for the lysosomal storage disease, infantile Neuronal ceroid lipofuscinosis (INCL) (Vesa et al., 1995), localization studies using mannose-6-phophate competition confirmed the lysosomal distribution of PPT1 (Hellsten et al., 1996; Verkruyse and Hofmann, 1996). PPT1 has been characterized as an enzyme that removes palmitate from specific cysteine residues in Ras, a protein known to be pro-proliferative and pro-tumor formation (Sellers and Fisher, 1999), so it could be postulated that PPT1 may be involved in the cancer pathway, possibly to inhibit growth or apoptosis. This role for PPT1 in cancer had not yet been explored prior to the work described herein.

Activation of Ras oncogenes occurs in a high percentage of human tumors, making the enzymes involved in the post-translational modification and membrane targetting of Ras, the target for anti-cancer drugs (Vojtek and Der, 1998). Attention initially focussed on the famesyltransferase (Haklai and Kloog, 1998) since its inhibition should reduce ras association with the membrane and hence its activation and subsequent tumor proliferation. Although such inhibitors do have anti-cancer activity it is not through their action on Ras since Ras can still be activated by geranylgeranylation even when famesylation is blocked. Though Ras is palmitoylated, which is required for Ras activation (Milligan et al., 1995), the focus is currently on alternative targets for famesylation (other than Ras) to explain the anti-cancer action of these drugs. In the past, many researchers considering palmitoylation as a target have assumed that increasing the level of Ras palmitoylation (say by blocking PPT1) would be pro-proliferative.

The antiproliferative agent Didemnin B uncompetitively inhibits palmitoyl protein thioesterase. Didemnin B, a cyclic depsipeptide isolated from a Caribbean sea squirt, was the first marine natural product to enter clinical trials as an anti-cancer agent and has advanced to phase II trials for a number of different human solid tumors based on its anti-proliferative action (Crews et al., 1996). DidemninB has several in vitro biological activities and was originally shown to bind to an activated translation elongation factor EF-1a. (Crews et al., 1996)—which is the likeliest explanation of its anti-proliferative activity. Subsequently, a second binding protein, PPT1 was identified as a binding partner of didemnin (Meng et al., 1998). Didemnin was then shown to non-competitively inhibit PPT1 activity as measured with both a [³H] Ha-Ras and a [³H]myristoylCoA in vitro assay. However, the relatively low affinity of the drug makes it highly toxic and of little value in inhibiting PPT1 in vivo. Also, Didemnin B-treated mice lacked the characteristic lysosomal accumulation of lipofuscin associated with INCL despite the lethal dosage administered. Further, there is a lack of correlation between PPT1 inhibitory Kᵢ values and IC₅₀ values reported for in vivo biological activities among different didemnins. Thus, the PPT1 inhibitory capacity of didemnin has nothing to do with its anti-proliferative action in vivo.

Lawrence et al. recently reported that the natural product cerulenin ([2R, 3S]-2,3-epoxy-4-oxo-7,10-trans, trans-dodecadienamide was able to inhibit the palmitoylation of both H-Ras- and N-Ras-encoded p21s in parallel with the inhibition of cell proliferation (Lawrence et al., 1999). Regression analyses indicated that inhibition of palmitoylation was more closely related to the inhibition of proliferation of T24 cells than was inhibition of fatty acid synthase activity, which would have a general effect on all lipid synthesis. They concluded that since activation of Ras oncogenes occurs in a high percentage of tumors, inhibitors of the palmitoylation of Ras could function as anti-tumor drugs.

Under this theory, because PPT1 is an enzyme that removes palmitate from Ras, it would not be considered a candidate anti-tumor drug. Moreover, PPT1's activity is more consistent with its being pro-proliferative and pro-tumor formation. Even though DidemninB has been identified as an anti-proliferative compound, this activity has not been tied to its ability to find PPT1 in a specific or competitive fashion. Therefore, insights into improved therapies for cancers based on PPT1 are needed. While there are available therapies for various cancers, including chemotherapy, radiotherapy, and immunotherapy, novel approaches showing improved success must be developed, and these could involve PPT1, which is involved in the Ras oncogene's activation pathway. Accordingly, the roles of post-translational modifiers of proteins involved in proliferation in cancer need to be addressed.

### SUMMARY OF THE INVENTION

The present invention is based on the observation that affecting PPT1, for example, by preventing or inhibiting its activity, can inhibit a cancer cell, such as by making the cell more susceptible to programmed cell death or apoptosis. Thus, the invention involves compositions and methods concerning PPT1 modulators, as well as methods for identifying such modulators. A “PPT1 modulator” refers to a compound that directly affects PPT1 expression, activity/function, or location, which means the compound interacts with a nucleic acid molecule encoding all or part of PPT1 (i.e., gene or transcript) or a PPT1 polypeptide or protein. A “PPT1 modulator” may be referred to with respect to any of its effects on PPT1, for example, as a “PPT1 modulator of PPT1 activity.” The PPT1 modulators described herein may be employed with any of the methods and compositions described herein. Furthermore, the methods and composition of the invention may be implemented with respect to a variety of cell and tissue types, as well as organisms. It is specifically contemplated that mammals, such as humans, may be the target of treatment or screening.

In some embodiments of the invention, methods of inhibiting a cancer cell can be effected by administering to the cancer cell a composition that includes a PPT1 modulator in an amount effective to reduce PPT1 activity level. It is contemplated that inhibiting a cancer cell may be accomplished by altering proliferation, metastasis, programmed cell death, contact inhibition, soft agar growth, cell cycle regulation, tumor formation, tumor progression, differentiation, or tumor invasion, though the invention is not limited to these ways. Furthermore, inhibiting a cancer cell may be achieved by altering the cell's susceptibility to programmed cell death, or apoptosis, including promoting apoptosis.

In other embodiments, methods of inhibiting a cancer cell may involve a modulator that competitively binds to PPT1. It is specifically contemplated that a modulator is a proteinaceous composition. In some embodiments, a proteinaceous composition competitively binds to PPT1. In other examples, the modulator may be, for example, an antagonist of PPT1, meaning the modulator interferes with the physiological activity of PPT1. In still further embodiments, the modulator decreases the amount of PPT1 protein, inhibits expression of PPT1, inhibits transcription of PPT1, or inhibits translation of PPT1.

The modulator may be a nucleic acid molecule, a polypeptide or protein, a peptide, or a small molecule that affects PPT1 activity, including a peptide mimetic. It is contemplated that any embodiment discussed herein with respect to a peptide may be applied to or with a peptide mimetic. In some embodiments, the modulator is at least one peptide or peptide mimetic that selectively interacts or binds with PPT1. The peptide may contain at most or at least 5 contiguous amino acids from SEQ ID NO:3, for example, the sequence VKIKK. In additional embodiments, the peptide is comprised of at most or at least 5 contiguous amino acids from SEQ ID NO:4., such as the sequence YCWLR. A peptide of less than 5 amino acids is contemplated, including one that is 3 or 4 amino acids in length. While in still further embodiments the peptide is DAP1. While in other cases, the PPT1 modulator is a polypeptide, such as an antibody against PPT1 or a polypeptide involved in post-translational modification of PPT1. A molecule with the sequence of SEQ ID NO:3 is contemplated to be in an amide form. It is further specifically contemplated that amide linkages in modulators of the invention are nonhydrolyzable, and thus, biodegradable. Additionally, it is contemplated that a modulator may be a peptide mimetic, including a peptide mimetic to the peptides disclosed herein.

Alternative embodiments include a PPT1 modulator that is a nucleic acid molecule that contains a promoter operably linked to a PPT1 gene segment. The PPT1 gene segment may be positioned in reverse orientation under the control of a promoter that directs expression of an antisense product. In other cases, the nucleic acid molecule encodes a ribozyme specific for an RNA transcript of PPT1 in a cell expressing an RNA transcript of PPT1. With nucleic acid modulators, it is contemplated that the nucleic acid segment is located on a vector.

With respect to any compounds or methods of the invention, it is further contemplated that the modulator will contain or be attached to a lipid component. In some cases, a peptide is attached to a lipid component e.g, DAP1 (AcG-palmitoyl diamino propionate-VKIKK) and may be termed “lipopeptide.” It is specifically contemplated that the attachment may be achieved though a non-hydrolyzable linkage, such as an amide linkage. In some embodiments a PPT1 modulator that is a peptide is attached to a lipid component. In some cases, the lipid component is a fatty acid, which may be unbranched or not. The lipid component may contain 6, 8, 10, 12, 14, 16, 18, 20, 22, 24, 26, 28, 30 or more carbon atoms in length. In some embodiments, the lipid component is 16 carbon atoms in length. In additional embodiments, modulators may be chemically modified. For example, a modulator may be prepared in an α-ketoamide form, such as with DAP1-ketoamide, where the ketone group is at C1 5 of the fatty acid. It is contemplated from the crystal structure of PPT1 that a better fit might be a lipid which has a double bond between C4 and C5 of the lipid component. Other structural modifications that introduce a bend or kink into the modulator may be implemented in modulators of the invention. Furthermore, an oxime ether in the lipid is further contemplated as a way of obtaining a more potent inhibitor of PPT1. It is also contemplated to replace the VKIKK peptide sequence with other amino acids and also with non-amino acids such as substituted benzylamines.

Methods of the invention may also include administration to the cancer cell a composition comprising a second anti-cancer agent, in addition to administering a PPT1 modulator. The anticancer agent may involve, surgery, chemotherapy, radiotherapy, immunotherapy, hormone therapy, or gene therapy. In some embodiments, the methods involve administration of a chemotherapeutic drug. It is contemplated that the chemotherapeutic drug is an alkylating agent, mitotic inhibitor, antibiotic, nitrosurea, antimetabolite, corticosteroid hormone, or other antineoplastic agent.

Other methods of the invention include treating a subject with cancer by administering to the subject a PPT1 modulator in an amount effective to inhibit a cancer cell in the subject, so that a therapeutic benefit is conferred on the subject. As discussed above, the PPT1 modulator may be any modulator disclosed, such as a peptide that selectively interacts with PPT1. Such a peptide may contain at least or at most 5 contiguous amino acids from SEQ ID NO:3, such as the sequence VKIKK. Alternatively, such a peptide may contain at least or at most 5 contiguous amino acids from SEQ ID NO:4, for example, the sequence YCWLR. The subject may be an mammal, such as a human. Additionally, peptide mimetics of sequences disclosed herein are contemplated as part of the invention. A peptide mimetic of VKIKK or YCWLR are modulators considered for use herein.

In addition to treatment methods, the present invention concerns methods of screening for substances with anti-cancer activity by determining whether a substance modulates PPT1. A compound that modulates PPT1 may affect, for example, its activity, such as rate or specificity; its expression, which includes its transcription or translation; its location; its post-translational processing; its availability; or its turnover rate, either at the nucleic acid or polypeptide level. Any of these modulators may cause changes to the following characteristics of a cancer cell: proliferation, metastasis, contact inhibition, soft agar growth, cell cycle regulation, tumor formation, tumor progression, and/or tumor invasion. In some embodiments of the invention, the method includes administering to a cancer cell a chemotherapeutic agent in addition to a candidate compound. It is contemplated that assaying for modulation of PPT1 may be achieved by assaying for the cancer cell's ability or susceptibility to undergo apoptosis. In these screening methods, a cell may be assayed for a characteristic prior to administration of the candidate substance and/or the cell may be assayed after administration of the candidate substance. Alternatively, a cell may be assayed for a particular characteristic after the candidate substance is administered while a different cell is assayed for the same characteristic in the absence of the candidate substance.

As with the therapeutic methods discussed above, a cell may be contacted or treated in vitro or in vivo. The cell may be in a mammal, for instance. It is further contemplated for use with any cancer cell, such as a melanoma, non-small cell lung, small-cell lung, lung, hepatocarcinoma, retinoblastoma, astrocytoma, glioblastoma, central nervous system, gum, tongue, leukemia, neuroblastoma, head, neck, breast, pancreatic, prostate, renal, bone, testicular, ovarian, mesothelioma, cervical, gastrointestinal, lymphoma, brain, colon, or bladder cell.

Furthermore, methods of evaluating a cell, tissue, organ, or subject for a condition or disease related to or affecting PPT1 are included in the present invention. Such conditions or diseases include pre-cancer or cancer. In some embodiments, the methods involve obtaining and evaluating a sample for PPT1 amount or activity level. A sample may include cells, tissue, organ, or bodily fluids such as blood, urine, and tears. PPT1 amount may be discerned by assaying PPT1 transcript or protein amounts. Alternatively, PPT1 activity or enzyme levels of the sample may be evaluated. The sample may be compared to a sample believed not to have or be subject to a particular condition or disease, such as cancer. Thus, in some embodiments of the invention, a sample is evaluated for a cancer by assaying PPT1 amount or activity level, and compared to a noncancerous sample. Any of the methods and disclosed herein may be implemented with respect to these diagnostic methods.

The invention also concerns pharmaceutical composition comprising a PPT1 modulator. In some embodiments of the invention, the composition includes a recombinant vector containing an PPT1 gene segment positioned in reverse orientation, under the control of a promoter that directs expression of an antisense product. While in other embodiments, the PPT1 modulator selectively binds to PPT1. In still further embodiments, a pharmaceutical composition includes a peptide covalently attached to a lipid component.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B. pH profile of in vitro PPT1 activity using various peptide substrates. PPT1 activity was measured at various pH using LA-N-5 cell extract. The data represent mean ±SEM from two experiments in duplicate.

FIG. 2A, FIG. 2B, FIG. 2C.

FIG. 2A. Loss of PPT1 activity in INCL lymphoblasts measured with Po and GAP43 peptides. PPT1 activity assayed using either Po (at pH 4.0, solid bar) or GAP43 (at pH 7.4, clear bar) demonstrated a significant loss of enzyme activity in INCL. CON, control lymphoblasts; CAR, carriers (BD 441 and 292) and INCL. Three INCL patients with identified mutations in PPT1 (BD437 (C451T, del 398T), BD442 (A364T, G550A) and BD445 (T29A, T29A)) were assayed (Das et al., 1998). The experiment was done twice in duplicate and data are mean ±SEM.

FIG. 2B. Extract from PPT1 overexpressing cells showed increased depalmitoylation of Po and GAP43 peptides. PPT1 activity was measured in extracts of either vector (Neo) or PPT1-transfected cells using either Po or GAP43 peptide as substrate. The data is a representative autoradiogram of HPTLC, showing greatly increased palmitate release in cells overexpressing PPT1. The experiment was done twice in duplicate with similar results.

FIG. 2C. [¹⁴C]Palmitoylated substrate peptides were alkaline-labile. Palmitoylated substrates were incubated in 6N NaOH or water for 30 min and subsequently Folch-extracted. Results showed that all radioactivity was converted to free palmitate in the presence of NaOH, indicating the alkaline-lability (thioester-nature) of these substrates.

FIG. 3A and 3B.

FIG. 3A. PPT1 inhibition by a substrate analogue. PPT1 activity was measured using LA-N-5 cell extract (at pH 4.0 for Po peptide substrate or at pH 7.4 for Gα, GAP43 and rhodopsin peptide substrates) following preincubation with various concentrations of AcG-palmitoyldiaminopropionate-VKIKK (DAP1) as described elsewhere in the application. The experiment was repeated twice with similar results.

FIG. 3B. Structure of AcG-palmitoyldiaminopropionate-VKIKK (DAP1).

FIG. 4. Treatment of LA-N-5 cells with C₂ ceramide or LY294002 results in apoptosis, accompanied by an increase in caspase-3 activity and DNA fragmentation in a time-dependent manner. Cells were treated with either C₂ ceramide (C2-Cer, 25 μM) or LY294002 (LY, 30 μM) for indicated times and assayed for cell viability (A), caspase 3 activity (B) or DNA fragmentation (C). The control group of cells was treated with vehicle (DMSO) for indicated times. Both C₂ ceramide and LY294002 induced cell death with time-dependent increase of caspase 3 activity and DNA fragments. The data represent mean ±SEM from at least two experiments done in duplicate.

FIG. 5A and FIG. 5B. Overexpression of PPT1 inhibited activation of caspase-3 by C₂ ceramide or LY294002. Vector alone (LAN) or PPT1 (PPT) transfected cells were treated with either C₂ ceramide (25 μM) or LY294002 (30 μM) and harvested at the indicated time. Aliquots of post-nuclear supernatant was assayed for caspase 3-like activity using DEVD-AFC as a substrate and the resultant fluorescence was quantified using the fluorometer at excitation 400 nm and emission 505 nm. Enzyme activity was calculated as the amount of fluorescence produced/mg protein/h. Experiments were done in duplicate at least twice and the data represent mean ±SEM. Results from PPT cells at all time points are statistically different from those of LAN (P<0.05).

FIG. 6. Reduced DNA fragmentation in PPT1-expressing cells following drug treatment. Vector (LAN) or PPT1 (PPT) transfected cells were treated with either C₂ ceramide (25 μM) or LY294002 (30 μM) for the time indicated and DNA fragments in the cell lysate (A) or the culture medium (B) was quantified using Hoechst 33285. The data represent mean ±SEM from at least two experiments done in duplicate. *p<0.05 compared with LAN.

FIG. 7. PPT1 overexpression resulted in increased cell survival following C₂ ceramide- or LY294002-induced apoptosis. Vector (LAN) or PPT1 (PPT) transfected cells were treated with either C₂ ceramide (25 μM) or LY294002 (30 μM) for 24 h or 48 h and cell viability was assessed by MTT assay. The data represent mean ±SEM from at least two experiments done in triplicate and all the PPT results are statistically different from LAN (p<0.05).

FIG. 8. Comparison of cell growth rate. Equal numbers (2.5×10⁴) of viable vector (NEO) or PPT1 (PPT) transfected cells were plated and grown in DMEM media containing 10% serum. At the indicated times, cells were trypsinized and the cell suspension counted for viable cells. The data represents the numbers from one experiment in duplicate, which was repeated three times with a similar result. *p<0.05 compared with NEO.

FIG. 9. Transfection of AS-PPT inhibits PPT1 activity. Protein extracts (50 μg) from control cells (CONT) or cells transfected with AS-PPT1 (AS) were assayed for PPT1 activity in vitro using either Po peptide or GAP43 peptide substrate as described in the text. The results represent mean +/−SEM of three individual assays done in triplicate.

FIG. 10. Inhibition of PPT1 enhances C₂-ceramide-induced killing of LAN-5 cells. Neo-transfected cells (A, NEO) or PPT1 overexpressing cells (B, PPT) were treated with increasing concentrations of the PPT1 inhibitor AcG-palmitoyl diamino propionate-VKIKK (DAP1) (0, 50 100 μM) for 7 h with (C2) or without (CONT) 30 μM C₂-ceramide. Cell viability was determined with the MTT assay as described in the text and results were expressed as percent cell death. For C₂-ceramide treatment, cells were preincubated with the PPT1 inhibitor for 1 h prior to the addition of C₂-ceramide.

FIG. 11A and FIG. 11B. Inhibition of PPT1 enhances etoposide killing of LAN-5 cells.

FIG. 11A. LAN-5 cells were treated with increasing concentrations of etoposide in the presence or absence of DAP1 (100 μM) for 24 h and cell viability determined by the MTT method.

FIG. 11B. Cell death induced by increasing concentrations of etoposide alone.

FIGS. 12A, 12B, 12C, 12D. DAP1 increases killing by four apoptotic drugs. LAN-5 cells were exposed to 100 μM of DAP1 (●) at various concentrations of either Etoposide, Daunorubicin, LY294002, or Staurosporine.

FIGS. 13A, 13B, 13C, 13D. PPT1 overexpression protects against killing by four apoptotic drugs. LAN-5 cells were transfected with PPT1 and then exposed to various concentrations of either Etoposide, Daunorubicin, LY294002, or C2-ceramide.

FIG. 14. PPT1 Substrates. Chemical structures of PPT1 substrates shown. Note that substrate is flurogenic substrate 4-methylumbelliferyl-beta-D-glucosyl-6-thio-palmitate. Glc indicates glucose.

FIGS. 15A-B. A. Relative Potency of DAP1 Compared to DAP1 ketoamide in killing HOG cells. B. Enhanced Killing of HOG Cells by DAP-KA When Added Together with Etoposide. Amount of DAP1 or DAP-KA is shown on the X-axis in μM.

FIG. 16. Non-peptide Inhibitors.

FIG. 17. Modification of the Lipid Moiety.

FIG. 18. α-Keto Heterocycle Inhibitors.

FIG. 19. Method for Synthesis of Keto Amides.

### DESCRIPTION OF ILLUSTRATIVE EMBODIMENTS

There is a need for new targets and new drugs in treating childhood cancers. Neuroblastoma is a common childhood cancer in which about 30% of cases have a very poor prognosis. While using cell lines derived from human neuroblastomas to investigate basic mechanisms of signal transduction in the nervous system a new class of target molecules for attacking cancer cells has been identified. The target molecule is an enzyme, palmitoyl:protein thioesterase (PPT), which removes palmitic acid residues from proteins and the drugs are inhibitors of this enzyme.

Protein palmitoylation occurs post-translationally, unlike the co-translational myristoylation and prenylation of proteins, and is a reversible modification with dynamic acylation/deacylation cycles. It results in the association of the protein with plasma membrane signalling molecules and is essential for the activity of many proteins associated with human cancers, such as the Ras family of GTP binding proteins and the Src family of tyrosine kinases. Inhibitors of Ras prenylation have been shown to have some efficacy as anti-cancer agents, but the finding that inhibition of PPT would increase the death of cancer cells was unexpected. In an Example, antisense DNA to PPT is shown to inhibit PPT activity and human neuroblastoma cells so treated underwent a more extensive cell death by apoptosis than untreated cells. Conversely, the overexpression of PPT protected the cells against apoptosis induced by a range of drugs such as staurosporine, PI-3 kinase inhibitors, ceramide, and serum-withdrawal. In order to see if PPT hydrolyzed intact palmitoylated proteins or could recognize a palmitoylated peptide fragment, a number of substrates were prepared in which radioactive ([3H]palmitic acid was linked either to peptide sequences asociated with palmitoylation sites or to cysteine in whole proteins. Palmitoylated peptides were found to be excellent and specific substrates for PPT. To test the idea more fully a non-hydrolyzable form of the K-Ras palmitoylated peptide was synthesized, which is called DAP1. This compound gave 85% inhibition at 20 μM in an in vitro assay. Current chemotherapeutic regimens for treating neuroblastoma include combinations of etoposide and carboplatin, doxorubicin-vincristine and cyclophosphamide. Treating intact LAN-5 human neuroblastoma cells with DAP1 did not result in much killing, but that, in combination with either etoposide or adriamycin, provided the same level of killing with only half the concentration of drug. This is extremely important because of the high cardiotoxicity of drugs such as adriamycin. DAP1 does not appear to be toxic to non-dividing cells and therefore has a very high probability of attacking only cancer cells.

DAP1 is toxic at high doses to other human cancer cells and its use as a combination drug may extend to many human cancers. In several different tumor cell lines, the drug was able to completely arrest cell division. These included breast cancer cell line T47D (minus 6%), prostate cancer cell line DU-145 (minus 4%), renal cancer cell line 498 (minus 31%), CNS cancer cell line SNB-19 (minus 11%) and non-small cell lung cancer cell line HOP-92 (minus 15%).

The present invention concerns the role of palmitoyl protein thioesterase 1 (PPT1) in cancer; it takes advantage of the observation that PPT1 renders a cancer cell more resistant to apoptosis and that modulation of PPT1 in a cancer cell promotes apoptosis. Promotion of apoptosis in cancer cells has significant implications for the treatment of cancer. Compositions that modulate PPT1 are contemplated for use in the treatment of cancer. Methods of the claimed invention involve screening for modulators of PPT1, as it was not previously known that such modulators resulted in the induction of apoptosis. Furthermore, the present invention includes treatment methods involving modulators of PPT1, which, in some cases, are administered in conjunction with another anti-cancer therapy such as chemotherapy.

I. Palmitoyl Protein Thioesterase (PPT) and PPT1 Modulators

A. Palmitoylation

Many proteins involved in a range of cellular functions are known to undergo co- or post-translational lipid modification to regulate their subcellular targetting and thereby the degree of activation (Mumby, 1997; James and Olson, 1990). Unlike myristoylation and prenylation, palmitoylation is a reversible post-translational modification with dynamic acylation/deacylation cycles (Bizzozero and Good, 1991; Magee et al., 1987), which further supports the physiological role of protein palmitoylation in regulating the signal transduction following ligand binding. For example, incorporation of palmitate into the α subunit of Gₛ protein has been shown to be enhanced when receptors were activated by a β-adrenergic agonist, via increased palmitoylation turnover (ref 36). Despite mounting evidence that palmitoylation of various proteins is involved in the regulation of biological functions, the enzyme responsible for palmitoylation has not been definitely identified as yet. The presence of acyltransferase or palmitoyl transferase activity has been reported (Schmidt et al., 1995; Berthiaume and Resh, 1995; Schroeder et al., 1996). However, other studies have demonstrated non-enzymatic acylation of myelin proteolipid protein, rhodopsin and Gα subunits (Bizzozero, 1997; Duncan and Gilman, 1996; O'Bried et al., 1987) as well as peptides (Bharadwaj and Bizzozero, 1995; Cho and Dawson, 1998) and both mechanisms may be of physiological significance.

Many proteins involved in signal transduction are palmitoylated, including several α subunits of G-proteins, G protein-coupled receptors such as adrenergic, serotonergic and dopaminergic receptors and rhodopsin, as well as cell-signaling molecules such as the Src family protein tyrosine kinases and p21^{Ras} proteins (Dunphy and Linder, 1998; Milligan et al., 1995). The sequence around the palmitoylated [P] cysteine residue appears to be conserved e.g., in kinases that are also myristoylated[M] at the N-terminal glycine (Gly). For example:

Fyn Met-[M]Gly-[P]Cys-Val-Gln-Cys-Lys-

Lck Met-[M]Gly-[P]Cys-Val-Cys-Ser-Ser-

Yes Met-[M]Gly-[P]Cys-Ileu-Lys-Ser-

Src Mer-[M]Gly-Ser-Ser-Lys-Ser-

The proteins Fyn, Lck, and Yes are all palmitoylated in vivo, while Src is not palmitoylated and in not found in the detergent-resistant membrane (drm) fraction. However, if the first Ser is replaced with Cys, it becomes palmitolyated in vivo and is located in the drm.

Neuron-specific hydrophilic proteins such as GAP-43 (neuromodulin), synaptosomal protein (SNAP-25) and postsynaptic density-95 protein (PSD-95), which are enriched at the synapse, are also known to be reversibly palmitoylated, and modulation of PPT1 as a result of a defective gene gives primarily neuronal symptoms. In myelin, Po glycoprotein and the proteolipid proteins are extensively acylated (Bizzozero, 1997; Bharadwaj and Bizzozero, 1995). The physiological role of attachment of long chain fatty acyl group to proteins has been postulated as being to control protein distribution between subdomains of plasma membrane and/or between membrane and cytoplasm. By concentrating signalling components such as the subunits of a K⁺ channel, this is believed to affect protein-protein interaction and modulates ligand-mediated activation of signaling cascades (Mumby, 1997; Milligan et al., 1995).

B. Palmitoyl Thioesterase

Palmitoyl protein thioesterase 1 (PPT1) was the first enzyme described to remove palmitate from lipid-modified proteins on specific cysteine residues. The initial enzyme assay to measure PPT activity employed [³H] palmitoylated Ras as a substrate (Camp and Hofinann, 1993), but the preparation of Ras by recombination technique, metabolic radiolabeling and purification of the radiolabeled protein has been too complicated for universal use as a routine clinical diagnostic procedure. In addition, intact Ras also seems to be hydrolyzed by another thioesterase activity present in lymphoblasts, as evidenced by the separation of two activities on Percoll gradient fractionation (Hofmann et al., 1997). PPT1 appears to be a typical lysosomal hydrolase and its deficiency results in the neurodegenerative disease of children, infantile Batten disease (INCL). The [³H]Ras assay showed the deficiency in these children but could not reliably detect heterozygote carriers of INCL in lymphocytes (Vesa et al., 1995), because of the presence of a second activity. A new assay using a [¹⁴C]pahnitoylated Po peptide as the substrate demonstrated no activity in patients with INCL and intermediate activity in heterozygotes (Cho and Dawson, 1998). The octapeptide substrate IRYCWLRR is first auto-acylated by incubation with [¹⁴C]palmitoyl CoA on the same site as is found in vivo (Cho and Dawson, 1998) and purified by HPTLC. Thus preparation is simple. Several different peptides can be used (Cho et al., 2000), suggesting that palmitoylated peptides rather than the intact protein may be the major physiological substrates for PPT1.

A second lysosomal thioesterase, PPT2, was subsequently cloned (Soyombo and Hofmann, 1997). PPT2 shows 18% identity to PPT1 at the amino acid level and shares such lysosomal hydrolase features as glycosylation, the presence of a signal peptide and uptake by mannose-6-phosphate receptor-mediated pathway with PPT1. PPT2 was able to hydrolyze palmitate from palmitoyl-CoA at a similar rate as PPT1, but was unable to remove palmitate from H-Ras or albumin or compensate for PPT1 deficiency. As expected, no mutations in the PPT2 gene have been found in INCL patients and the addition of PPT2 did not prevent the accumulation of [35S] cysteine-labeled storage materials in INCL lymphoblasts.

Acyl-protein thioesterase (APT), which is palmitoyl thioesterase 3, is a cytosolic enzyme that can remove palmitate from the Ga subunit and H-Ras (Duncan and Gilman, 1998). It was previously characterized as a lysophospholipase (Sugimoto et al., 1996) and shows enzyme activity toward both lysophosphocholine and palmitoyl-Gᵢₐ, although the latter appears to be a preferred substrate for APT in vitro. Overexpression of APT by stable transfection into mammalian cells resulted in a maximum two to three fold increase in enzyme activity, similar to that observed for PPT1 overexpression in neuroblastoma cells (Cho and Dawson, 2000). There was a moderate increase in the rate of turnover of palmitate in G_{sα} in vivo as a result of APT overexpression. APT is also unable to compensate for the defect in INCL and its role as a major deacylating enzyme in cells is unclear. It appears from all these studies that a tight control over the balance and turnover of protein thioacylation is crucial to maintain cellular function and integrity. This idea is supported by a report of the developmental regulation of PPT1 in neurons (Suopanki et al., 1999a; Suopanki et al., 1999b). In most cases, palmitoylation is the signal for membrane attachment of a protein that has been previously myristoylated at an N-terminal glycine residue or prenylated at the C-terminus (Verheij et al., 1998; Vojtek and Der, 1998).

PPT1 was initially identified as a cytosolic lipase that removes palmitate from activated Ras and an α-subunit of Go protein (Camp and Hofmann, 1993). Following its identification as the gene responsible for the lysosomal storage disease infantile Neuronal ceroid lipofuscinosis (INCL) (Vesa et al., 1995), localization studies using mannose-6-phophate competition confirmed the lysosomal distribution of PPT1 (Hellsten et al., 1996; Verkruyse and Hofmann, 1996). In COS cells, the uptake of exogenously supplied PPT1 was blocked by mannose-6-phosphate and the classic Finnish mutation (A122W) disrupted intracellular routing of PPT1 to the lysosome with retention of protein in the endoplasmic reticulum. The demonstration of an acidic pH optimum for PPT1 in brain and in lymphoblasts (Soyombo and Hofmann, 1997) supports the lysosomal identity of PPT1. Further, percoll gradient analysis of MDCK cells showed that PPT activity primarily co-resided with a lysosomal enzyme marker (Verkruyse and Hofmann, 1996).

Despite these lines of evidence that the primary localization of PPT1 is the lysosome, other observations suggest that the role of PPT1 is not limited to lysosomes. Thus although PPT1 activity overlapped with a lysosomal marker enzyme more than other particulate fractions (e.g., mitochondria, Golgi or endosome), the relative distribution of enzyme activity between cytosolic and particulate compartments has not been reported, and the majority (80%) of PPT activity was originally reported in the cytosolic fraction (Cho et al., 2000). Discrepancies in the pH optimum range from acidic (pH 4-5) (Cho and Dawson, 1998) to neutral (pH 7) (Camp et al., 1994), suggests a complex nature for the physiological role of PPT1. Studies suggest a different pH optimum of PPTl towards different peptide substrates (Cho et al., 2000).

PPT1 was initially described as an enzyme that removes palmitate from specific cysteine residues in Ras and by analogy from other palmitoylated proteins (57). Based on current dogma that membrane-bound Ras is pro-proliferative (Sellers and Fisher, 1999) and pro-tumor formation (Sellers and Fisher, 1999), it was expected that overexpressing PPT1 (and thereby decreasing palmitoylation) would enhance cell death by apoptosis. Apoptosis is a central mechanism for programmed death of cells during tissue remodeling in development and decreased apoptosis can result in tumorigenesis, making the apoptotic signalling pathway a target for cancer drugs (Sellers and Fisher, 1999). Essentially all animal cells have ability to undergo apoptosis by activating an intrinsic cell suicide program (Jacobson et al., 1997; Steller, 1998) which leads to morphologically distinct changes (Kerr et al, 1972) characterized by cell shrinkage, chromatin condensation, membrane blebbing and intemucleosomal DNA fragmentation (Arends and Wylie, 1991; Krueger et al., 1995). Biochemically, the final stages of apoptosis are characterized by the activation of a unique class of cysteine-aspartic acid proteases termed caspases, which act on key nuclear proteins such as poly (ADP-ribose) polymerase (Sellers and Fisher, 1999; Salvesen and Dixit, 1997). Proteolysis is followed by endonuclease cleavage of DNA into 180-200 bp fragments (DNA ladder) and eventual absorption of apoptotic bodies by macrophages (Arends and Wylie, 1991; Krueger et al., 1995). Gene products that induce or protect against apoptosis have been identified in simple nervous systems and many of the mammalian homologues have been identified. For example, in C. elegans, ced-9 is the equivalent of the mammalian protective bcl-2 gene (Martinou et al., 1994; Allsopp et al., 1993; Shimizu et al., 1995) and bcl2/bcl_{XL} family members are increased in proportion to the metastatic potential of the tumor cell (Coffer et al., 1998). Drugs discussed below that are used chemotherapeutically to increase tumor apoptosis, such as etoposides and daunorubicin, have been shown to act by increasing the levels of the sphingolipid, ceramide (a family of N-acylsphingosines) (Goswami et al., 1998; Goswami et al., 1999).

The present invention is directed at compounds and methods involving compounds that specifically modulate PPT1. These PPT1 modulators may act in any way to specifically prevent, reduce, or delay PPT1 activity, such as by binding PPTI. A “PPT1 modulator” refers to a compound that specifically and/or competitively modulates PPT1, which means the compound specifically and/or competitively affects PPT1 activity, directly or indirectly. As discussed below, these modulators may be comprised of nucleic acids, amino acids, small molecules, or a combination thereof.

C. Proteinaceous Compounds

In certain embodiments, the present invention concerns compositions comprising at least one proteinaceous molecule. The compound may include a recombinant PPT1 protein (SEQ ID NO:2), or it may include a proteinaceous molecule that is involved in the modulation of PPT1. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein. Furthermore, these terms may be applied to fusion proteins as well.

In certain embodiments the size of at least one proteinaceous molecule may comprise-but is not limited to-about, at least, or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 350, 400, 450, 500, 550, 600, 650, 700, 750, 800, 850, 900, 950, 1000, 1100, 1200, 1300, 1400, 1500, 1750, 2000, 2250, 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| | | | |
|---|---|---|---|
| Abbr. | Amino Acid | Abbr. | Amino Acid |
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic | AHyl | allo-Hydroxylysine |
| | acid | | |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Accordingly, the term “proteinaceous composition” encompasses amino molecule sequences comprising at least one of the 20 common amino acids in naturally synthesized proteins, or at least one modified or unusual amino acid.

1. Functional Aspects

The present invention concerns PPT1, particularly recombinant PPT1 and amino acid molecules that modulate PPT1, which is a protein that removes palmitate from specific cytsteine residues of particular proteins. Modulation of PPT1 has an anti-proliferative effect on a cell expressing PPT1 and/or renders the cell more susceptible to being killed by another anti-cancer therapy such as a chemotherapeutic drug. Thus, when the present application refers to the function or activity of PPT1 or a “PPT1 polypeptide,” one of ordinary skill in the art would understand that this includes, for example, the ability to remove a palmitate molecule. On the other hand, when the present invention refers to the function or activity of a “PPT1 modulator,” one of ordinary skill in the art would further understand that this includes, for example, the ability to specifically or competitively bind PPT 1 or an ability to promote cell death in a cell that expresses PPT 1. Determination of which molecules are suitable modulators of PPT1 may be achieved using assays familiar to those of skill in the art-some of which are disclosed herein and may include, for example, the use of native and/or recombinant PPT1.

PPT1 modulators that are polypeptides may include any polypeptide that affects PPT1, for example, those polypeptides that inhibit its ability remove palmitate from Ras. Such polypeptides would include Ras family members and neuron-specific palmitoylated proteins, such as GAP-43, SNAP-25, and PSD-95.

2. Variants of PPT1

Amino acid sequence variants of the polypeptides and peptides of the invention—PPT1 and modulators of PPT1—can be substitutional, insertional or deletion variants. These include polymorphisms and mutants that affect the activity of PPT1 or of modulators of PPT1, such as their ability to competitively bind PPT1.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a PPT1 polypeptide are included, provided the biological activity of the protein is maintained.

The term “functionally equivalent codon” is used herein to refer to codons that encode the same amino acid, such as the six codons for arginine or serine, and also refers to codons that encode biologically equivalent amino acids (see Table 2, below).

TABLE 2
CODON TABLE

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, binding sites to substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides and peptides according to the invention is the use of peptide mimetics. Mimetics are peptide-containing molecules that mimic elements of protein secondary structure. See, e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of a PPT1 modulator, but with altered and even improved characteristics.

3. Peptide Sequences

As a way of effecting modulation of PPT1, small peptides or fusion peptides may be implemented, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length.

Peptides that cause a cell to undergo cell death or be susceptible to drugs that induce cell death may be employed. For example, short peptides have been designed that have two functional domains, one a tumor blood vessel “homing” motif such a cyclic CNGRC and the other a programmed cell death-inducing sequence such as the D-enantiomer of KLAKLAKKLAKLAK connected by a GG bridge (Ellerby et al., 1999). The peptides induced apoptosis in cell lines. The peptides were then tested in vivo in nude mice with human MDA-MD-435 breast carcinoma xenografts and found that tumor volume was reduced to 10% of untreated and that 60% were still alive after 100 days compared to none of the untreated. No apparent toxicities were found in mice after 3 months at drug levels of 250 μg/mouse/week. Although the compounds of the present invention appear to bear no relationship to these drugs, the basic principal is the same. Further if there are problems with drug uptake a targetting peptide sequence could be implemented to get the drug into the cell.

It is further understood that peptides that bind to PPT1 are clearly included in the invention, such as those that specifically, selectively, and/or competitively bind to PPT1.

This would include peptides corresponding to one or more antigenic determinants of the PPT1 polypeptide; these peptides can be prepared so that an immune response against PPT1 is raised.

U.S. Pat. No. 4,554,101, (Hopp) incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the PPT1 sequence.

Numerous scientific publications have also been devoted to the prediction of secondary structure, and to the identification of epitopes, from analyses of amino acid sequences (Chou & Fasman, 1974a,b; 1978a,b, 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson & Wolf, 1988; Wolf etal., 1988), the program PepPlot® (Brutlag et al, 1990; Weinberger et al., 1985), and other new programs for protein tertiary structure prediction (Fetrow & Bryant, 1993). Another commercially available software program capable of carrying out such analyses is MacVector (IBI, New Haven, Conn.).

In further embodiments, major antigenic determinants of a PPT1 polypeptide may be identified by an empirical approach in which portions of the gene encoding the PPT1 polypeptide are expressed in a recombinant host, and the resulting proteins tested for their ability to elicit an immune response. For example, PCR™ can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTsTM system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR™ cloning methodology.

4. Lipid Components and Moieties

In certain embodiments, the present invention concerns compositions comprising one or more lipids associated with a nucleic acid, an amino acid molecule, such as a peptide, or another small molecule compound. In any of the embodiment discussed herein, the molecule may be either PPT1 or a PPT1 modulator, for example a nucleic acid encoding all or part of either PPT1 or a PPT1 modulator, or alternatively, a amino acid molecule encoding all or part of PPT1 modulator. A lipid is a substance that is characteristically insoluble in water and extractable with an organic solvent. Compounds than those specifically described herein are understood by one of skill in the art as lipids, and are encompassed by the compositions and methods of the present invention. A lipid component and a non-lipid may be attached to one another, either covalently or non-covalently.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

a. Lipid Types

A neutral fat may comprise a glycerol and/or a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from at least 2, 3, 4, 5, 6, 7, 8, 9, 10, 11, 12, 13, 14, 15, 16, 17, 18, 19, 20, 21, 22, 23, 24, 25, 26, 27, 28, 29, to 30 or more carbon atoms, and any range derivable therein. An example of a range is from about 8 to about 16 carbon atoms in the chain portion of the fatty acid. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated. The fatty acid may be branched, though in embodiments of the present invention, it is unbranched.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phoshotidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

b. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

c. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

d. Lipid Composition Structures

A nucleic acid molecule or amino acid molecule, such as a peptide, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid/PPT1 modulator-associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-PPT1 modulator or Superfect (Qiagen)-PPT1 modulator complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10%to about 35% phosphatidyl choline, and about 1% of a drug. Thus, it is contemplated that lipid compositions of the present invention may comprise any of the lipids, lipid types or other components in any combination or percentage range.

i. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

ii. Micelles

A lipid may be comprised in a micelle. A micelles is a cluster or aggregate of lipid compounds, generally in the form of a lipid monolayer, may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

e. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a lipid and/or PPT1 modulator may be, for example, encapsulated in the aqueous interior of a liposome, interspersed within the lipid bilayer of a liposome, attached to a liposome via a linking molecule that is associated with both the liposome and the PPT1 modulator, entrapped in a liposome, complexed with a liposome, etc.

i. Making Liposomes

A liposome used according to the present invention can be made by different methods, as would be known to one of ordinary skill in the art. For example, a phospholipid (Avanti Polar Lipids, Alabaster, AL), such as for example the neutral phospholipid dioleoylphosphatidylcholine (DOPC), is dissolved in tert-butanol. The lipid(s) is then mixed with the PPT1 modulator, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the PPT1 modulator is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of modulatory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000× g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiements, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/PPTl modulator or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. Liposomal encapsulation has resulted in a lower toxicity and a longer serum half-life for such compounds (Gabizon et al., 1990).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

ii. Liposome Targeting

Although targetting may be achieved by employing a particular peptide sequence, association of the PPT1 modulator with a liposome may also improve biodistribution and other properties of the PPT1 modulator. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/PPT1 modulator composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of PPT1 modulator. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

5. Biochemical Cross-Linkers

It can be considered as a general guideline that any biochemical cross-linker that is appropriate for use in an immunotoxin will also be of use in the present context, and additional linkers may also be considered to join proteinaceous compositions that include peptides and polypeptides of the present invention.

Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation.

TABLE 3
HETERO-BIFUNCTIONAL CROSS-LINKERS

| | | | Spacer Arm |
|---|---|---|---|
| | | | Length/after cross- |
| linker | Reactive Toward | Advantages and Applications | linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Suithydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It can therefore be seen that a targeted peptide composition will generally have, or be derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking. For a general overview of linking technology, one may wish to refer to Ghose & Blair (1987).

The spacer arm between the two reactive groups of a cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e.g., disulfide bond resistant to reducing agents). The use of peptide spacers, such as L-Leu-L-Ala-L-Leu-L-Ala, is also contemplated.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagents for use in immunotoxins is SMPT, which is a bifunctional cross-linker containing a disulfide bond that is “sterically hindered” by an adjacent benzene ring and methyl groups. It is believed that stearic hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the bispecific coagulating ligands of this invention.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art.

Once conjugated, the peptide generally will be purified to separate the conjugate from unconjugated targeting agents or coagulants and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

In addition to chemical conjugation, a PPT1 modulator or PPT1 polypeptide, peptide, or antibody may be modified at the protein level. Included within the scope of the invention are IgA protein fragments or other derivatives or analogs that are differentially modified during or after translation, for example by glycosylation, acetylation, phosphorylation, amidation, derivatization by known protecting/blocking groups, and proteolytic cleavage. Any number of chemical modifications may be carried out by known techniques, including but not limited to specific chemical cleavage by cyanogen bromide, trypsin, chymotrypsin, papain, V8 protease, NaBH₄; acetylation, formylation, famesylation, oxidation, reduction; metabolic synthesis in the presence of tunicamycin.

6. Immunological Reagents

In certain aspects of the invention, one or more antibodies may be produced to polypeptides, proteins, and peptides. These antibodies may be used in various diagnostic or therapeutic applications, described herein below.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow and Lane, 1988, incorporated herein by reference).

Monoclonal antibodies (MAbs) are recognized to have certain advantages, e.g., reproducibility and large-scale production, and their use is generally preferred. The invention thus provides monoclonal or polyclonal antibodies of the human, murine, monkey, rat, hamster, rabbit and even chicken origin. “Humanized” antibodies are also contemplated, as are chimeric antibodies from mouse, rat, or other species, bearing human constant and/or variable region domains, bispecific antibodies, recombinant and engineered antibodies and fragments thereof.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, Pa.); low-dose Cyclophosphamide (CYP; 300 mg/m²) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial inimunoglobulin phagemid libraries are prepared from RNA isolated from the spleen of the immunized animal, and phagemids expressing appropriate antibodies are selected by panning using cells expressing the antigen and control cells.

Alternatively, monoclonal antibody fragments encompassed by the present invention can be synthesized using an automated peptide synthesizer, or by expression of full-length gene or of gene fragments in E. coli.

a. Antibody Conjugates

The present invention further provides antibodies to ORF-transcribed messages and translated proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al, 1995; Cleary et al., 1994; Lenert et al, 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins.”

Antibody conjugates may be employed for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium^{188,} ⁷⁵selenium, ³⁵sulphur, technicium^{99m} and/or yttrium⁹⁰, ¹²⁵I, is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulffydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

b. Immunodetection Methods

In still further embodiments, the present invention concerns immunodetection methods for binding, purifying, removing, quantifying and/or otherwise generally detecting biological components such as ORF expressed message(s), protein(s), polypeptide(s) or peptide(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle MH and Ben-Zeev O, 1999; Gulbis B and Galand P, _{—}1993; De Jager R et al., 1993; and Nakamura et al., 1987, each incorporated herein by reference.

In general, the immunobinding methods include obtaining a sample suspected of containing ORF expressed message and/or protein, polypeptide and/or peptide, and contacting the sample with a first anti-ORF message and/or anti-ORF translated product antibody in accordance with the present invention, as the case may be, under conditions effective to allow the formation of immunocomplexes.

These methods include methods for purifying an ORF message, protein, polypeptide and/or peptide from organelle, cell, tissue or organism's samples. In these instances, the antibody removes the antigenic ORF message, protein, polypeptide and/or peptide component from a sample. The antibody will preferably be linked to a solid support, such as in the form of a column matrix, and the sample suspected of containing the ORF message, protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the ORF produced antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ORF antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The ORF antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various diseases wherein a specific ORF is expressed, such as an viral ORF of a viral infected cell, tissue or organism; a cancer specific gene product, etc. Here, a biological and/or clinical sample suspected of containing a specific disease associated ORF expression product is used. However, these embodiments also have applications to non-clinical samples, such as in the titering of antigen or antibody samples, for example in the selection of hybridomas.

In the clinical diagnosis and/or monitoring of patients with various forms a disease, such as, for example, cancer, the detection of a cancer specific ORF gene product, and/or an alteration in the levels of a cancer specific gene product, in comparison to the levels in a corresponding biological sample from a normal subject is indicative of a patient with cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention in any immunodetection or therapy known to one of ordinary skill in the art.

i. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-ORF message and/or anti-ORF translated product antibodies of the invention are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another anti-ORF message and/or anti-ORF translated product antibody that is linked to a detectable label. This type of ELISA is a simple “sandwich ELISA.” Detection may also be achieved by the addition of a second anti-ORF message and/or anti-ORF translated product antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with the anti-ORF message and/or anti-ORF translated product antibodies of the invention. After binding and/or washing to remove non-specifically bound immune complexes, the bound anti-ORF message and/or anti-ORF translated product antibodies are detected. Where the initial anti-ORF message and/or anti-ORF translated product antibodies are linked to a detectable label, the immune complexes may be detected directly. Again, the immune complexes may be detected using a second antibody that has binding affinity for the first anti-ORF message and/or anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

ii. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

c. Immunodetection Kits

In still further embodiments, the present invention concerns immunodetection kits for use with the immunodetection methods described above. As the PPT1 and/or PPT1 modulator antibodies are generally used to detect wild-type and/or mutant PPT1 and/or PPT1 modulator proteins, polypeptides and/or peptides, the antibodies will preferably be included in the kit. However, kits including both such components may be provided. The immunodetection kits will thus comprise, in suitable container means, a first antibody that binds to a wild-type and/or mutant PPT1 and/or PPT1 modulator protein, polypeptide and/or peptide, and/or optionally, an immunodetection reagent and/or further optionally, a wild-type and/or mutant PPT1 and/or PPT1 modulator protein, polypeptide and/or peptide.

In preferred embodiments, monoclonal antibodies will be used. In certain embodiments, the first antibody that binds to the wild-type and/or mutant PPT1 and/or PPT1 modulator protein, polypeptide and/or peptide may be pre-bound to a solid support, such as a column matrix and/or well of a microtitre plate.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

The kits may further comprise a suitably aliquoted composition of the wild-type and/or mutant PPT1 and/or PPT1 modulator protein, polypeptide and/or polypeptide, whether labeled and/or unlabeled, as may be used to prepare a standard curve for a detection assay. The kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. Where wild-type and/or mutant PPTI and/or PPT1 modulator protein, polypeptide and/or peptide, and/or a second and/or third binding ligand and/or additional component is provided, the kit will also generally contain a second, third and/or other additional container into which this ligand and/or component may be placed. The kits of the present invention will also typically include a means for containing the antibody, antigen, and/or any other reagent containers in close confinement for commercial sale. Such containers may include injection and/or blow-molded plastic containers into which the desired vials are retained.

D. Nucleic Acid Molecules

1. Polynucleotides Encoding PPT1 or a PPT1 Modulator

The present invention concerns polynucleotides, isolatable from cells, that are free from total genomic DNA and that are capable of expressing a protein or polypeptide that is derived from the PPT1 gene product or a PPT1 modulator gene product. Polynucleotides of the invention also concern molecules that are not translated into a polypeptide, but whose activity serves to modulate PPT1, such as PPT1 ribozymes and antisense constructs. Any of the methods discussed herein with regard to PPT1 may be applied to any PPT1 modulator, including peptides that modulate PPT1. Recombinant PPT1 or PPT1 modulator can be purified from expressing cells to yield active PPT1 or PPT1 modulator. Thus, it is contemplated that a polypeptide or peptide of the invention, such as PPT1 or a PPT1 modulator, may be produced recombinantly.

As used herein, the term “DNA segment” refers to a DNA molecule that has been isolated free of total genomic DNA of a particular species. Therefore, a DNA segment encoding a PPT1 polypeptide refers to a DNA segment that contains wild-type, polymorphic, or mutant PPT1 polypeptide-coding sequences yet is isolated away from, or purified free from, total mammalian or human genomic DNA, for example SEQ ID NO: 1, which is the human cDNA sequence encoding PPT1. Included within the term “DNA segment” are a polypeptide or polypeptides, DNA segments smaller than a polypeptide, and recombinant vectors, including, for example, plasmids, cosmids, phage, viruses, and the like.

As used in this application, the term “polynucleotide” refers to a nucleic acid molecule that has been isolated free of total genomic nucleic acid. Therefore, a “polynucleotide encoding a PPT1 polypeptide” or a “polynucleotide encoding a PPT1 modulator” refers to a DNA segment that contains wild-type, polymorphic, or mutant PPT1 polypeptide-coding sequences, yet is isolated away from, or purified free from, total mammalian or human genomic DNA. Therefore, when the present application refers to the function or activity of PPT1 or “PPT1 polypeptide” that is encoded by a PPT1 polynucleotide, it is meant that the polynucleotide encodes a molecule that has the ability to remove, for example, palmitate from particular cysteine residues of Ras. Once again, any of the methods and compositions disclosed herein may be applied with respect to a PPT1 modulator, that is, polypeptide or peptide molecule functions to modulate PPT 1 and prevent its activity.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given PPT 1 from a given species may be represented by natural variants that have slightly different nucleic acid sequences but, nonetheless, encode the same protein (see Table 1 above). Consequently, the present invention also encompasses derivatives of a PPT1 polypeptide that have minimal amino acid changes, but that possess the palmitoyl protein thioesterase properties of PPT1 and/or its ability to inhibit apoptosis.

Similarly, a polynucleotide comprising an isolated or purified wild-type, polymorphic, or mutant PPT1 polypeptide gene refers to a DNA segment including wild-type, polymorphic, or mutant PPT1 polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid encoding PPT1, or alternatively a PPT1 modulator, may contain a contiguous nucleic acid sequence encoding all or a portion of PPT1, or a PPT1 modulator, of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500 or more nucleotides, nucleosides, or base pairs.

In particular embodiments, the invention concerns isolated DNA segments and recombinant vectors incorporating DNA sequences that encode a wild-type, polymorphic, or mutant PPT1 polypeptide or peptide that includes within its amino acid sequence a contiguous amino acid sequence in accordance with, or essentially corresponding to wild-type, polymorphic, or mutant PPT1 polypeptides.

In other embodiments, the invention concerns isolated DNA segments and recombinant vectors incorporating DNA sequences that encode a PPT1 polypeptide or peptide that includes within its amino acid sequence a contiguous amino acid sequence in accordance with, or essentially corresponding to the PPT1 polypeptide.

The nucleic acid segments used in the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length PPT1 from any source or encode a truncated version of PPT1, such that the transcript of the coding region represents the truncated version. The truncated transcript may then be translated into a truncated protein. Alternatively, a nucleic acid sequence may encode a full-length PPT1 protein sequence with additional heterologous coding sequences, for example to allow for purification of PPT1, transport, secretion, or post-translational modification of PPT1. As discussed above, a tag may be added to the PPT1-encoding sequence.

In a non-limiting example, one or more nucleic acid constructs may be prepared that include a contiguous stretch of nucleotides identical to or complementary to the PPT1 gene. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent PPT1 polypeptides and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein or to test mutants in order to examine DNA binding activity at the molecular level.

2. Vectors

PPT1 or a PPT1 modulator may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding PPT1, a vector may encode non-PPT1 sequences such as a tag. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCRTM, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Tables 4 lists several elements/promoters that may be employed, in the context of the present invention, to regulate the expression of a gene. This list is not intended to be exhaustive of all the possible elements involved in the promotion of expression but, merely, to be exemplary thereof. Table 5 provides examples of inducible elements, which are regions of a nucleic acid sequence that can be activated in response to a specific stimulus.

TABLE 4
Promoter and/or Enhancer

| Promoter/Enhancer | References |
|---|---|
| Inmmnoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| Promoter/Enhancer | References |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake |
| | et al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Chol et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; Rosen |
| | et al., 1988; Berkhout et al., 1989; Laspia et al., 1989; |
| | Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 5
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1 987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T | Taylor et al., 1989, 1990a, 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al, 1998), mouse alpha2 (XI) collagen (Tsumaki, et al, 1998), DIA dopamine receptor gene (Lee, et al., 1997), insulin-like growth factor II (Wu et al., 1997), human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a PPT1-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURER Competent Cells and SOLOPACK™ Gold Cells (STRATAGENE®, La Jolla). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBA® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'S COMPLETE CONTROL™ Inducible Mammalian Expression System, which involves a synthetic ecdysone-inducible receptor, or its pET Expression System, an E. Coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

5. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

6. Methods of Gene Transfer

Suitable methods for nucleic acid delivery to effect expression of PPT1 for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau etal., 1987; Wong etal., 1980; Kaneda etal., 1989; Kato etal., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirullehetal., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

7. Antisense Constructs

Antisense constructs targetting PPT1 transcript or PPT1 genomic sequences may be used in any of the methods of the present invention. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is altered.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct that has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

8. Ribozymes

Similar to the use of antisense constructs, ribozymes targetting PPT1-encoding sequences may be employed in any of the methods of the present invention. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al, 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

9. Nucleic Acid Detection

In addition to their use in directing the expression of PPT1 modulator proteins, polypeptides and/or peptides, the nucleic acid sequences disclosed herein have a variety of other uses. For example, they have utility as probes or primers for embodiments involving nucleic acid hybridization. They may be used in diagnostic or screening methods of the present invention. Detection of nucleic acids encoding PPT1 or PPT1 modulators are encompassed by the invention.

a. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to SEQ ID NO:1 or any other SEQ ID NO are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

c. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCRTM (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

e. Kits

All the essential materials and/or reagents required for detecting part or all of SEQ ID NO:1 or any PPT1 modulator in a sample may be assembled together in a kit. This generally will comprise a probe or primers designed to hybridize specifically to individual nucleic acids of interest in the practice of the present invention, including SEQ ID NO: 1. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

II. Treatment of Cancer

A. Treatment of Cancer or Precancer

The present invention involves the treatment of cancer and precancer/preneoplastic conditions. The types of conditions that may be treated, according to the present invention, are limited only by the involvement of a PPT1 modulator. By involvement, it is meant that a PPT1-modulator inhibits a cancer cell or a tumor. The term “cancer cell” is used to indicate a cell whose growth is uncontrolled. In addition to cancers where a tumor is not formed, it is contemplated that a wide variety of tumors, including solid tumors, may be treated using PPT1-modulator or anti-PPT1 therapy, including cancers of the brain (glioblastoma, astrocytoma, oligodendroglioma, ependymomas), lung, liver, spleen, kidney, lymph node, pancreas, small intestine, colon, stomach, breast, endometrium, prostate, testicle, ovary, skin, head and neck, esophagus, bone marrow, blood and other tissue. Moreover, the treatment of pre-neoplastic conditions is also included since precancerous lesions can lead to the development of cancer. These pre-neoplastic conditions include, for example, oral hairy leukoplasia, bronchial dysplasia, carcinomas in situ, and intraepithelial hyperplasia.

In many contexts, it is not necessary that the cell be killed or induced to undergo normal cell death or “apoptosis.” Rather, to accomplish a meaningful treatment involving a PPT1 modulator, all that is required is that the growth of cancer cells or tumor growth be slowed to some degree. It may be that the cell's growth is completely blocked, however, or that some tumor regression is achieved. Clinical terminology such as “remission” and “reduction of tumor” burden also are contemplated given their normal usage.

The term “therapeutic benefit” used throughout this application refers to anything that promotes or enhances the well-being of the subject with respect to the medical treatment of his condition, which includes treatment of pre-cancer and cancer. A list of nonexhaustive examples of a “therapeutic benefit” includes extension of the subject's life by any period of time, decrease or delay in the neoplastic development of the disease, decrease in growth or proliferation of cancer cells, reduction in tumor growth, delay or prevention of metastases, reduction in cancer cell or tumor cell proliferation rate, and a decrease in pain to the subject that can be attributed to the subject's condition.

In order to increase the effectiveness of an anti-cancer therapy, it may be desirable to combine a PPT1-modulator with an anti-cancer agent as a combination treatment. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

D. Combined Therapy with Immunotherapy, Traditional Chemotherapy, Radiotherapy or Other Anti-Cancer Agents

Tumor cell resistance to DNA damaging agents represents a major problem in clinical oncology. One goal of current cancer research is to find ways to improve the efficacy of chemo- and radiotherapy. One way is by combining therapies with agents, such as a PPT1 modulator—for example, DAP1—that increase the effectiveness of existing therapies and/or reduce their side effects. For example, the herpes simplex-thymidine kinase (HS-tk) gene, when delivered to brain tumors by a retroviral vector system, successfully induced susceptibility to the antiviral agent gancyclovir (Culver et al., 1992). In the context of the present invention, it is contemplated that PPT1 modulator therapy could be used similarly in conjunction with anti-cancer agents, including chemo- or radiotherapeutic intervention. It also may prove effective to combine troglitozone with immunotherapy that targets cancer/tumor cells.

To kill cells, inhibit cell growth, inhibit metastasis, inhibit angiogenesis or otherwise reverse or reduce the malignant phenotype of cancer cells, using the methods and compositions of the present invention, one would generally contact a “target” cell with a PPT1 modulator and at least one other agent. These compositions would be provided in a combined amount effective to kill or inhibit proliferation of the cell. This process may involve contacting the cells with the PPT1 modulator and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a PPT1 modulator or the other agent will be desired. Various combinations may be employed, where a PPT1 modulator is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Again, to achieve cell killing, both agents are delivered to a cell in a combined amount effective to kill the cell.

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

B. Chemotherapeutic Agents

A wide variety of chemotherapeutic agents may be used in combination with the use of a PPT1-modulator or anti-PPT1 agent in the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas. It is contemplated that PPT1 modulators can be used in combination with one or more of these agents according to the present invention.

1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

a. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

b. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal 10 half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with a PPT1 modulator in the treatment of cancer.

c. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

d. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

e. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

a. 5-Fluorouracil

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

a. Doxorubicin

Doxorubicin hydrochloride, 5,12-Naphthacenedione, (8 s-cis)-10-[(3-amino-2,3,6-trideoxy-a-L-lyxo-hexopyranosyl)oxy]-7,8,9,1 0-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations. It is also called Adriamycin.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

Myocardial toxicity manifested in its most severe form by potentially fatal congestive heart failure may occur either during therapy or months to years after termination of therapy. The probability of developing impaired myocardial function based on a combined index of signs, symptoms and decline in left ventricular ejection fraction (LVEF) is estimated to be 1 to 2% at a total cumulative dose of 300 mg/m² of doxorubicin, 3 to 5% at a dose of 400 mg/m², 5 to 8% at 450 mg/m² and 6 to 20% at 500 mg/m². The risk of developing CHF (congestive heart failure) increases rapidly with increasing total cumulative doses of doxorubicin in excess of 450 mg/m². This toxicity may occur at lower cumulative doses in patients with prior mediastinal irradiation or on concurrent cyclophosphamide therapy or with pre-existing heart disease. In the present invention the inventors have employed a PPT1 modulator to synergistically enhance the antineoplastic effects of the doxorubicin in the treatment of cancers. Use of DAP1 would enable doxorubicin to be used at levels that would not cause cardiotoxicity yet still have the same efficacy. Those of skill in the art will be able to use the invention as exemplified potentiate the effects of doxorubicin in a range of different pre-cancer and cancers.

b. Daunorubicin

Daunorubicin hydrochloride, 5,12-Naphthacenedione, (8S-cis)-8-acetyl-10-[(3-amino-2,3 ,6-trideoxy-a-L-lyxo-hexanopyranosyl)oxy]-7,8 ,9,1 0-tetrahydro-6,8, 11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m2 /day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

c. Mitomycin

Mitomycin (also known as mutamycin and/or mitomycin-C) is an antibiotic isolated from the broth of Streptomyces caespitosus which has been shown to have antitumor activity. The compotmd is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Corticosteroid Hormones

Corticosteroid hormones are useful in treating some types of cancer (lymphoma, leukemias, and multiple myeloma). Though these hormones have been used in the treatment of many non-cancer conditions, they are considered chemotherapy drugs when they are implemented to kill or slow the growth of cancer cells. Like DAP 1, corticosteroid hormones can increase the effectiveness of other chemotherapy agents, and consequently, they are frequently used in combination treatments. Prednisone and dexamethasone are examples of corticosteroid hormones.

5. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP 16), paclitaxel, taxol, vinblastine, vincristine, and vinorelbine.

a. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/ m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

b. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

c. Vinblastine

Vinblastine is another example of a plant aklyloid that can be used in combination with a PPT1 modulator for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m, 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

d. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can alos be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

6. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

a. Carmustine

Carmustine (sterile carmustine) is one of the nitrosoureas used in the treatment of certain neoplastic diseases. It is 1,3 bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. The structural formula is:

Sterile carmustine is commonly available in 100 mg single dose vials of lyophilized material. Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m², 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject.

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

7. Miscellaneous Agents

Some chemotherapy agents do not qualify into the previous categories based on their activities. However, it is contemplated that they are included within the method of the present invention for use in combination therapies of cancer with a PPT1 modulator. They include amsacrine, L-asparaginase, tretinoin, and Tumor Necrosis Factor (TNF), some of which are discussed below.

a. Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

C. Radiotherapy

Other factors that cause DNA damage and have been used extensively include what are commonly known as y-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

D. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could also be used as part of a combined therapy, in conjunction with Ad-mda7 gene therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine pro-apoptotic effect, mediated by PPT1 modulator treatment with immune stimulatory effects. However, alternate immune stimulating molecules also exist including: cytokines such as IL-2, IL-4, IL-12, GM-CSF, gamma-IFN, chemokines such as MIP-1, MCP-1, IL-8 and growth factors such as FLT3 ligand.

1. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

2. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

3. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

E. Genes

In yet another embodiment, the secondary treatment is gene therapy in which a therapeutic polynucleotide is administered before, after, or at the same time as a PPT1 modulator or a molecule encoding such modulator. This will have a combined anti-hyperproliferative effect on target tissues. Alternatively, a single vector encoding both nucleic acid molecules may be used. A variety of proteins are encompassed within the invention, some of which are described below. Various genes are listed below that may be targeted for gene therapy of some form in combination with the present invention.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein Rasfrom proto-oncogene to oncogene, in one example, results from a valine to glycine mutation at amino acid 12 in the sequence, reducing RasGTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is pl6. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al, 1994; Kamb et al., 1994; Kamb et al., 1994; Mori et al., 1994; Okamoto et al., 1994; Nobori et al, 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, Al, Bfl-1) or counteract Bcl-2 function and promote cell death (e.g., Bax, Bak, Bik, Bim, Bid, Bad, Harakiri).

F. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

G. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

H. Pharmaceutical Compositions

Aqueous compositions of the present invention comprise an effective amount of the PPT1 modulator directed therapeutic dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains an RBP agent as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A PPT1-directed agent of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like also can be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

F. in vitro, ex vivo, in vivo Administration

As used herein, the term in vitro administration refers to manipulations performed on cells removed from an animal, including, but not limited to, cells in culture. The term ex vivo administration refers to cells that have been manipulated in vitro, and are subsequently administered to a living animal. The term in vivo administration includes all manipulations performed on cells within an animal.

In certain aspects of the present invention, the compositions may be administered either in vitro, ex vivo, or in vivo. U.S. Pat. Nos. 4,690,915 and 5,199,942, both incorporated herein by reference, disclose methods for ex vivo manipulation of blood mononuclear cells and bone marrow cells for use in therapeutic applications.

In vivo administration of the compositions of the present invention also is contemplated. Examples include, but are not limited to, chemotherapy of bladder epithelium by administration of the chemotherapeutic compositions of the present invention through intravesicle catheterization into the bladder (Bass et al., 1995), and chemotherapy of liver cells by infusion of appropriate chemotherapeutic compositions through the portal vein via a catheter (Bao et al., 1996). Additional examples include direct injection of tumors with the instant compositions, and either intranasal or intratracheal (Dong et al., 1996) instillation of chemotherapeutic compositions to effect transduction of lung cells.

1. Therapeutically Effective Amounts of a PPT1 Modulator

A therapeutically effective amount of a PPT1 modulator, which may be combined with a second agent as treatment, varies depending upon the host treated and the particular mode of administration. In one embodiment of the invention the dose range of an emodin-like tyrosine kinase inhibitor used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, lmg/kg body weight to 450 mg/kg body weight, 2 mg/kg body weight to 400 mg/kg body weighty, 3 mg/kg body weight to 350 mg/kg body weighty, 4 mg/kg body weight to 300 mg/kg body weight, 5 mg/kg body weight to 250 mg/kg body weighty, 6 mg/kg body weight to 200 mg/kg body weight, 7 mg/kg body weight to 150 mg/kg body weighty, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10, mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for a PPT1 modulator alone or for such a compound in combination with an anti-cancer drug.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as a PPT1 modulator for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, the PPT1 modulator will be administered in combination with a second agent. So long as a dose of second agent that does not exceed previously quoted toxicity levels is not required, the effective amounts of the second agents may simply be defined as those amounts effective to reduce the cancer growth when administered to an animal in combination with the PPT1 modulating agents. This is easily determined by monitoring the animal or patient and measuring those physical and biochemical parameters of health and disease that are indicative of the success of a given treatment. Such methods are routine in animal testing and clinical practice.

Chemotherapy is typically administered in regular cycles. A cycle may involve one dose, after which several days or weeks without treatment ensues for normal tissues to recover from the drug's side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type and stage of cancer) or may be flexible, in order to take into account how quickly the tumor is shrinking. Certain serious side effects may also require doctors to adjust chemotherapy plans to allow the patient time to recover.

IV. Screening For Modulators of PPT1

The present invention further comprises methods for identifying modulators of PPT1, including those that inhibit its activity or function. These assays may comprise random screening of large libraries of candidate substances, such as random peptide libraries; alternatively, the assays may be used to focus on particular classes of compounds selected with an eye towards structural attributes that are believed to make them more likely to modulate PPT1. It is contemplated the PPT1 modulation may be assayed by the following ways, though the invention is not limited to these ways: 1) measuring, identifying, and/or characterizing an interaction between a candidate compound and PPT1; 2) measuring and/or characterizing an effect of the candidate compound on the activity or function of PPT1, such as its ability to remove a palmitate moelcule from a polypeptide substrate; or 3) measuring, identifying, and/or characterizing an effect on a cell containing PPT1, for example, the ability of that cell to be inhibited, for example, to undergo apoptosis.

By activity or function, it is meant that one may assay for a measurable effect on PPTl's ability to remove palmitate from a polypeptide, such as Ras. To identify a PPT1 modulator, one generally will determine characteristics of a cell in the presence and absence of the candidate substance, wherein a modulator is defined as any substance that alters these characteristics. For example, a method may generally comprise:

(i) contacting a first cancer cell with the candidate substance; and

(ii) comparing one or more characteristics of the first cell in the presence of the candidate substance with one or more characteristics of a second cancer cell in the absence of the candidate substance.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially selectively or competitively or specifically interact with PPT 1, which includes molecules that specifically inhibit or enhance its activity. The candidate substance may be a protein or peptide, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one that exerts its effect upstream, downstream or directly on PPT1. Regardless of the type of modulator identified by the present screening methods, the effect of such a compound results in alteration in PPT1 activity or specificity as compared to that observed in the absence of the added candidate substance.

2. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Depalmitoylation of Peptides by PPT1

A. Materials and Methods

1. Materials

[1-¹⁴C]palmitoyl CoA (59 mCi/mmol) was purchased from Amersham, Arlington Heights, Ill. Synthetic peptides Po (IRYCWLRR), 4Po (RYCW), rhodopsin (VTTLCCGKN), GAP43 (MLCCMRR), Gαs (MGCLGNSK) and H-Ras (GCMSCKCVLS) were purchased from Research Genetics, Huntsville, Ala. The peptide sequence selected was based on the palmitoylation motif of endogenous proteins (Bizzozero, 1997). The inhibitor peptide (AcG-palmitoyl diamino propionate-VKIKK) and its base peptide (AcGCVKIKK) was synthesized as descibed below. Boc-Dap(Fmoc) was from Bachem Bioscience (King of Prussia, Pa.), hydrogen fluoride was from Matheson Gas (Cucamonga,Calif.) and amino acids from Midwest Biotech (Ind.). Tfx-50 transfection reagent was from Promega (Madison, Wis.) and solvents were ACS grade from Fisher Scientific, Pittsburgh, Pa.

2. Peptide inhibitor synthesis

The peptides AcGCVKIKK and AcG (palmitoyl diaminoproprionate-VKIKK were synthesized by solid phase peptide synthesis using in situ neutralization cycles for Boc chemistry (Schnolzer et al., 1992). The peptides were synthesized on a 0.2 mmol scale on 4-methylbenzhydrilamine resin using 1.0 mmol of each activated amino acid and 20 min coupling times. The peptide was acetylated by treating the neutralized peptide resin with 2×5 mL of 0.5 M acetic anhydride, 0.5 M pyridine in N,N-dimethylformamide for one min. Diaminoproprionic acid (Dap) was incorporated as Boc-Dap(Fmoc)-OH using standard coupling conditions. Following chain assembly, the Fmoc group was removed with 20% piperidine in DMF for 10 min and the resulting free amine was acylated with 1.0 mmol palmitoyl chloride in 2 ml methylene chloride for 1 h. The peptides were deprotected and cleaved from the resin using 10 ml hydrogen fluoride and 5% v/v p-cresol as a scavenger. Following removal of the hydrogen fluoride under vacuum, the peptide was washed, precipitated with ether and dissolved in 6M guanidine hydrochloride, 100 mM sodium acetate buffer, pH 5.0. The peptides were immediately purified by reverse phase HPLC and lyophilized.

3. Cell Culture

LA-N-5 human neuroblastoma cells were grown in monolayers on 100 mm diam. tissue culture dishes in Dulbecco's modified Eagle's medium (DMEM) supplemented with 10% fetal bovine serum and 1% gentamycin. Immortalized lymphoblastoid cells were obtained from the BDSRA/NIH Mutant Cell Repository at the Indiana University School of Medicine in Indianapolis. Cells were grown in suspension in 75 cm² T-flasks in RPMI 1640 culture medium supplemented with 10% fetal bovine serum and 1% gentamycin. The diagnosis of INCL was made by clinical history, electron microscopic identification of granular osmophilic deposits, genetic analysis and enzyme assay of PPT1 (Das et al., 1998).

4. Preparation of [¹⁴C]palmitoylated substrates

Palmitoylation of peptides was performed as described previously with slight modifications (Cho and Dawson, 1998; Bharadwaj and Bizzozero, 1995). Fifty μg of each synthetic peptide (IRYCWLRR (Po), RYCW (4Po), VTTLCCGKN (rhodopsin), MLCCMRR (GAP43), MGCLGNSK (Gα) or GCMSCKCVLS (H-Ras)) was incubated with approximately 2 nmol of [1-¹⁴C]pahnitoyl CoA (59 mCi/mmol) in 0.1 M 3-[N-morpholino]propanesulfonic acid (MOPS) buffer (pH 7.4) containing 1 mM DTT and 0.1% Triton X-100. The reaction was stopped by immediately placing the tube at −20° C. Palmitoyl peptides were separated by HPTLC using n-butanol/pyridine/acetic acid/water (45:30:9:36, v/v). After developing the autoradiogram, the radioactive spot corresponding to the palmitoyl peptide was extracted and reconstituted in 25% methanol except the GCMSCKCVLS reaction mixture, which was unable to be resolved by HPTLC.

5. Measurement of PPT1 Activity

PPT1 activity was measured as described by Cho and Dawson (1998). In brief, 50 μg of cell extract was incubated with [¹⁴C] palmitoyl peptides (4000-5000 cpm) in 50 mM sodium citrate buffer (pH 4.0) for Po and 4Po or in 50 mM Tris (pH 7.4) for GAP43, rhodopsin and Ga unless indicated otherwise. Incubations were performed for 20 min at 37° C. and reactions terminated by addition of 1 ml of chloroform/methanol/2N HCl (2:1:0.06, v/v). After the centrifugation, organic phase was analyzed by HPTLC using n-butanol/pyridine/acetic acid/water (45:30:9:36, v/v) and [14C] palmitate release was quantified. In the study using PPT1 substrate analogue to inhibit enzyme activity, cell extract was preincubated with various concentrations of the inhibitor peptide for 15 min at 37° C. and enzyme reaction was initiated by adding the indicated substrate as described above.

6. PPT1 Plasmid Construction and Transfection

Preparation of the pcDNA3.1-PPT1 construct and stable expression in LA-N-5 cells were as described previously (Cho and Dawson, 2000). In brief, cDNA for human PPT1 prepared by reverse-transcription polymerase chain reaction was cloned into pcDNA3.1 using Kpn I/Xba I multiple cloning site. The correct insertion of cDNA was verified by sequence analysis and used for transfection of cells. Transfection of human PPT1 gene or vector alone was done using Tfx-50 transfection reagent according to the manufacturer's instructions, followed by the selection in the presence of G418 (500 μg/ml) for 3 weeks and cloning of individual cells.

B. Palmitoylation of Different Peptide Substrates

In order to see whether PPT1 exhibits differential capability to depalmitoylate different peptide substrates, several palmitoylated synthetic peptides were employed in an in vitro PPT assay. The sequences were chosen from known in vivo palmitoylation sites. Palmitoylated peptide substrates were prepared by incubating synthetic peptides with [¹⁴C] palmitoyl CoA as a donor, similar to the method used for Po octapeptide (Cho and Dawson, 1998). Upon incubation, all peptides underwent spontaneous [¹⁴C] palmitoylation. The palmitoylated peptides were isolated from the reaction mixture by HPTLC, except H-Ras peptide. [¹⁴C] H-Ras peptide could not be resolved by HPTLC even after trying several solvent systems (data not shown).

C. PPT Activity Does not Depend on Peptide Length.

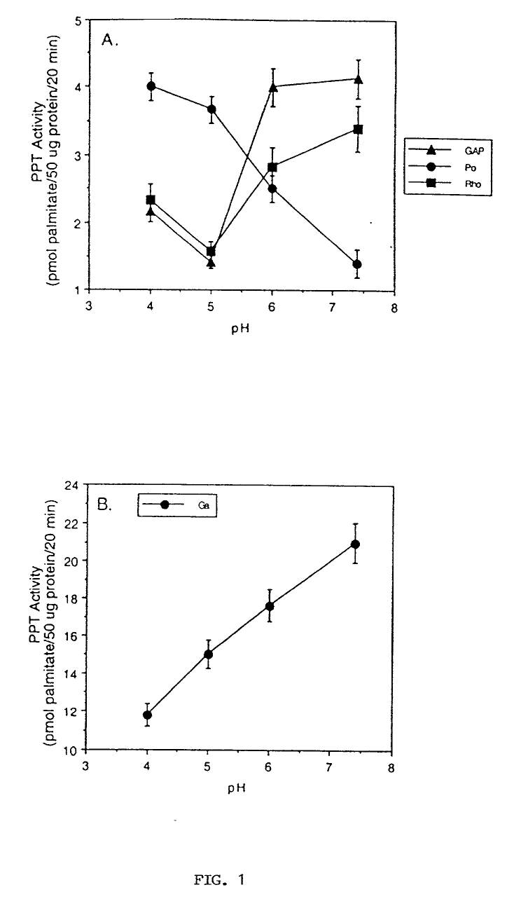

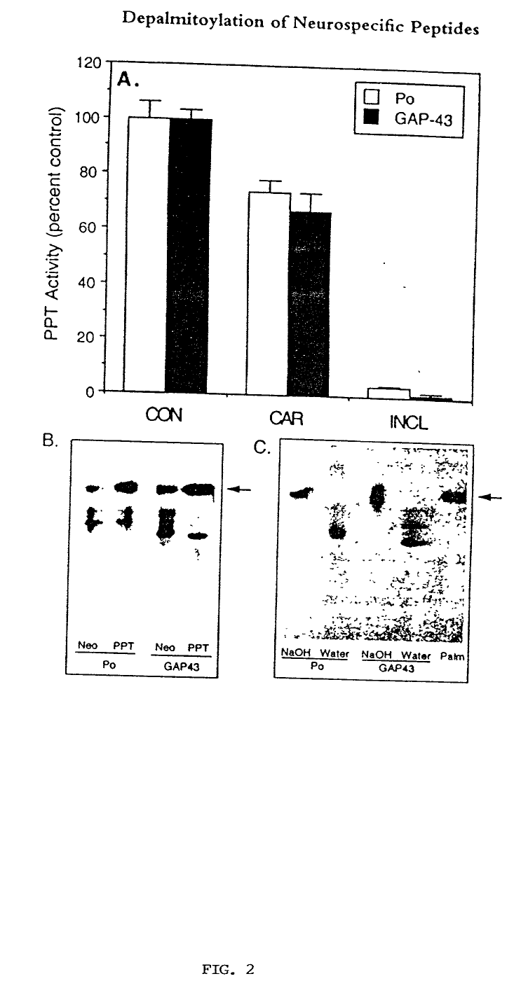

Using these substrates, PPT activity was measured in extracts from human neuroblastoma cells, LA-N-5, and LA-N-5 in which PPT1 was overexpressed. Po octapeptide was depalmitoylated by PPT1 with an acidic optimum pH (FIG. 1A) as described previously (Cho and Dawson, 1999). In order to see the effect of size of peptide substrate on enzymatic efficiency, we prepared a shorter sequence of Po (tetrapeptide, 4Po) and compared PPT activity measured with Po and 4Po. It was found that 4Po peptide was deacylated at the same rate as Po and with the same pH profile (data not shown).

D. Different Peptide Substrates Show Different pH Profiles

Other palmitoylated substrates derived from the appropriate sequences of neuron-specific GAP-43, retinal-specific rhodopsin and ubiquitous Gα peptides were tested. All were depalmitoylated by LA-N-5 extracts, but the depalmitoylation profile of these substrates was drastically different from that of Po. Thus GAP-43 and rhodopsin demonstrated a biphasic pH profile with higher activity at both acidic and neutral pH (FIG. 1A). In contrast, Gα peptide was most efficiently depalmitoylated at the neutral pH with more than 5 times higher efficiency that the rest of the substrates (FIG. 1B). Removal of palmitate from [¹⁴C]-palmitoyl-GAP-43 peptide was only 1-2% of control in extracts of lymphoblasts from INCL patients with verified PPT deficiency. Thus the GAP-43 peptide substrate used in these assays is depalmitoylated only by PPT1 (FIG. 2A) and not some other protein with depalmitoylating activity such as PPT2 or APT). Cells from heterozygotes showed about 70% of control PPT activity with the GAP-43 substrate, which is comparable to Po.

E. Overexpressed PPT hydrolyses the peptide substrates

Other evidence supporting the contention that PPT1 is the major enzyme that depalmitoylates these peptide substrates was obtained by using cells specifically overexpressing PPT1. The same amount of crude extract from PPT1- or vector transfected cells was used for PPT assay using peptide substrates. As shown in FIG. 2B, depalmitoylation of GAP-43 was increased approximately 2.5-fold in cells with PPT overexpression, and an overall three to four fold increase in depalmitoylation was observed with all peptide substrates. This further supports the validity of these palmitoylated compounds as useful substrates in assessing PPT activity in vitro. The thioester linkage in these substrates was alkaline-labile as expected, and treatment at basic pH (>10) resulted in the release of palmitate in the absence of any cell extract (FIG. 2C).

F. Synthesis of a Specific Inhibitor of PPT

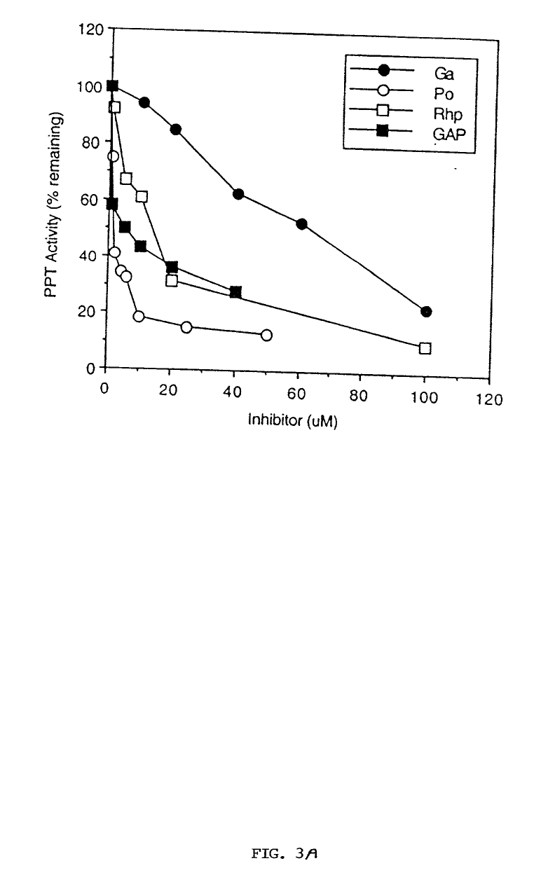

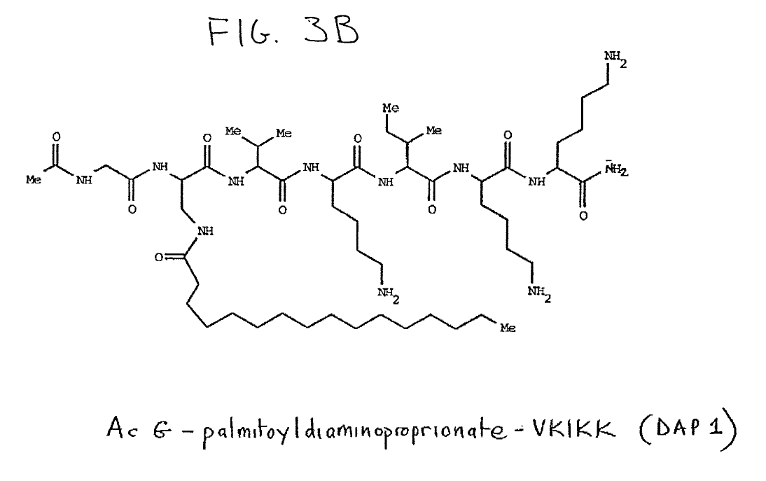

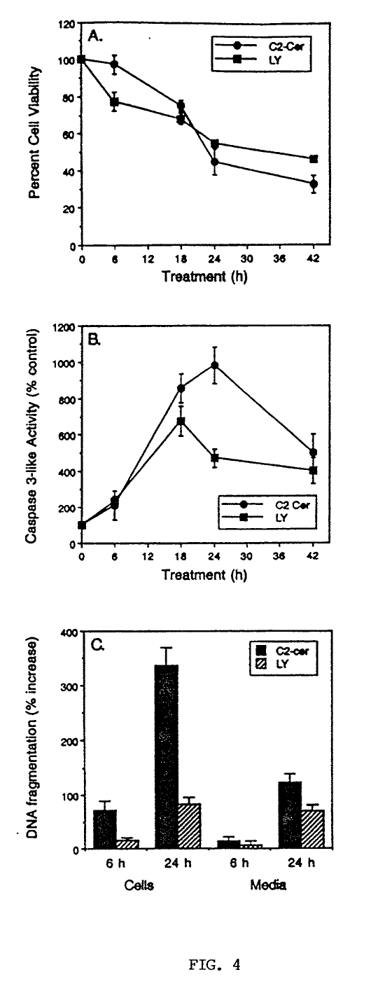

Based on the capability of PPT1 to depalmitoylate peptides where palmitate is linked to a cysteine residue via a thioester bond, that a non-hydrolyzable palmitoylated peptide analogue would block PPT activity was investigated. The peptide sequence, AcGCVKIKK (a palmitoylation site in K-Ras), was modified by substituting the SH group of cysteine with NH2 to generate diamino propionic acid, which was then acylated with palmitate (AcG-palmitoyl diamino propionate-VKIKK). This results in a CONH amide linkage instead of a COS thioester linkage. PPT activity was measured using indicated substrates in a standard way as described above, except that the cell extract was preincubated with various concentrations of the analogue substrate for 15 min at 37° C. prior to the addition of radiolabeled substrate. As shown in FIG. 3, the presence of the amide substrate efficiently inhibited PPT1 activity. The inhibition was more potent towards Po, rhodopsin and GAP-43 peptides than towards Gα peptide. In another set of experiments, the unmodified peptide itself (AcGCVKIKK) was used as a control to exclude the possibility of a nonspecific effect of the sequence on PPT activity. Preincubation of cell extract with AcGCVKIKK itself did not affect PPT activity toward any of the substrates tested.

### Example 2

### PPT1 Protects Against Apoptosis

A. Materials and Methods

1. Materials

[1-¹⁴C]palmitoyl CoA (59 mCi/mmol), [³²p] orthophosphate (1 Ci/mmol) and [³⁵S] cysteine (300 Ci/mmol) were purchased from Amersham Life Science (Arlington Heights, Ill.). LY294002 and DEVD-amino trifluoromethyl coumarin (AFC) were purchased from Biomol Research Labs (Plymouth Meeting, Pa.), C₂-ceramide from Matreya Inc. (Pleasant Gap, Pa.), MTT, Hoechst 33285, pFLAG-CMV-5a and anti-GAP-43 from Sigma (St. Louis, Mo.), Super signal ECL detection kit from Pierce (Rockford, Ill.), monoclonal anti-Ras, anti-caspase 3 antibody and protein A/G-conjugated agarose from Santa Cruz Biotechnology (Santa Cruz, Calif.), anti-phosphoserine Akt antibody from New England Biolabs (Beverly, Mass.), and Akt1 and Akt2 antibodies from Upstate Biotechnology (Lake Placid, N.Y.). Silica gel high performance thin layer chromatography (HPTLC) plates were from Whatman (Clifton, N.J.), the BioRad protein assay kit from Biorad (Hercules, Calif.), Tfx-50 transfection reagent and TNT kit from Promega (Madison, Wis.), and Trizol from Life Technologies (Gainthersburg, Md.). The chloroform, methanol, butanol, pyridine and acetic acid used for HPTLC were ACS grade from Fisher Scientific (Pittsburgh, Pa.).

2. Preparation of hPPT1 cDNA and Plasmid Construction

cDNA for human PPT1 gene was prepared by reverse-transcription polymerase chain reaction using RNA isolated from LA-N-5 cells. The first strand cDNA was amplified with a forward primer:

forward primer:
5′TCTAGGTACCAAGATGGCGTCGCCCGGCTGCCTGT 3′
and a reverse primer:
5′ACGGTCTAGATCATCCAAGGAATGGTATGATGTGGGCA 3′

to be subsequently cloned into the expression vector. After digestion, the entire amplified sequence was inserted into the multiple cloning site of pcDNA 3.1. The correct insertion of cDNA was verified by sequence analysis and used for transfection of cells. For in vitro translation of PPT1, the construct was incubated with T7-RNA polymerase-coupled TNT kit in the presence of [³⁵S] cysteine according to the manufacturer's protocol and the product analyzed by 10% SDS electrophoresis. FLAG-tagged PPT1 was prepared by inserting PPT1 into C-terminal pFLAG-CMV-5a expression vector.

3. Cell Culture and Transfection

LA-N-5 human neuroblastoma cells were grown in monolayers on 100 mm diameter tissue culture dishes in Dulbecco's modified Eagle's medium (DMEM) supplemented with 10% fetal bovine serum and 1% gentamycin. Transfection of human PPT1 gene or vector alone was done using Tfx-50 transfection reagent according to the manufacturer's instruction. To establish stably transfected clones, cells were selected by growing in media containing G418 (500 μg/ml) for 3 weeks, and individual clones were isolated. At the time of drug treatment, cells were washed with serum-free media and incubated with serum-free media containing LY294002 or C₂ ceramide until harvested.

4. PPT1 Assay

PPT1 activity was measured as described previously (Cho and Dawson, 1998). In brief, the cell sonicate was incubated in the assay mixture containing 50 mM sodium citrate (pH 4.0) and IRY([⁴C]palmitoylated)CWLRR octapeptide (2,000-4,000 cpm) for 20 min at 37° C. For the pH profile of PPT1 activity, 50 mM sodium citrate (pH 5 or 6) or 50 mM Tris-HCl (pH 7.4) was used with other conditions remaining the same. After addition of 1 ml of chloroform/methanol/2N HCl (2:1:0.06, by volume) and centrifugation, the organic phase was dried, reconstituted sample analyzed by HPTLC using n-butanol/pyridine/acetic acid/water (45:30:9:36, by volume) and the radioactive spot corresponding to [¹⁴C] palmitate was counted.

5. Cell Viability Assay

Cells were plated in 24-well culture plates containing 500 μl media. After drug treatment, 50 μl MTT (5 mg/ml in PBS) was added and incubated for 20 min at 37° C. An aliquot of 500 μl of 10% sodium dodecyl sulfate in 0.01 N HCl was added. After incubating overnight, the absorbance at 570 nm was determined by spectrophotometry. Cell viability was calculated as percent cell survival by dividing absorbance [treatment-blank] by absorbance [control-blank], where treatment is the reading for the treated cells, control is for vehicle-treated cells and blank is for MTT added to the media without cells. We have previously demonstrated that MTT results correlate well with cell counts and DNA laddering (Wiesner and Dawson, 1996).

6. DNA Fragmentation Assay

Quantification of DNA fragments was performed as described previously (Dawson et al., 1997). Briefly, cells were lysed in 5 mM Tris (pH 8.0) with 0.1% Triton X-100 and 20 mM EDTA and centrifuged at 20,000× g for 40 min at 4° C. For determination of DNA fragments lost in culture media, an aliquot of media was adjusted to a final concentration of 25 mM EDTA and centrifuged as described above. Aliquots of the supernatants were added to the assay buffer consisting of 100 mM Tris (pH 7.4), 2 M NaCl, 10 mM EDTA, pH 7.4, and 0.1 ng/ml Hoechst 33285 (Labarca and Paigen, 1980). Fluorescence was detected by luminescent spectrometer using excitation 365 nm and emission 460 mn. DNA values were calculated using a standard curve obtained from the measurement of calf thymus DNA fluorescence.

7. Measurement of Caspase 3-like Activity

Cells treated with drug or vehicle were harvested, washed with PBS and cell pellets lysed in 25 mM Hepes (pH 7.4), containing 2 mM dithiothreitol (DTT), 5 mM EDTA and 10 mM digitonin. After centrifugation at 6,000 rpm for 10 min to remove cell debris and unruptured cells, an aliquot of the supernatant was added to a reaction buffer consisting of 25 mM Hepes (pH 7.4), 2 mM DTT, 5 mM EDTA, 10 mM digitonin and 2.5 μg/ml DEVD-AFC. After 1 h incubation, the hydrolysis of substrate was quantified by fluorometry at excitation 400 mn and emission 505 nm. The blank reaction was done in the absence of cell lysate. Enzyme activity was expressed as the amount of fluorescence produced per mg protein/h.

8. Western Blot Analysis

Cells were harvested in a lysis buffer containing 10 mM Tris (pH 7.4), 1 mM EGTA, 1 mM EDTA, 1 mM DTT, 25 mM NaF, 2 mM MgCl₂ and 10 μg/ml aprotinin, leupeptin and pepstatin. For subcellular fractionation, the postnuclear cell extract was centrifuged at 100,000× g for 45 min to obtain a cytosolic fraction and the pellet was washed and resuspended in a lysis buffer to obtain a membrane fraction. Whole cell lysate (for caspase-3 and pFLAG-PPT1) or fractionated proteins (for p₂₁Ras and GAP-43) were resolved by 10%, 12% or 15% of SDS-PAGE for GAP-43, caspase-3 and pFLAG-PPT1, or Ras, respectively. For immunoblotting with anti-Akt antibodies, cells were lysed in RIPA buffer consisting of 50 mM Tris (pH 7.4), 150 MM NaCl, 1 mM EGTA, 1% Nonidet P-40, 0.25% sodium deoxycholate, 1 mM NaF and protease inhibitors. After lysis on ice for 1 h, cell debris was removed by centrifugation at 3,000 rpm for 5 min and an equal amount of postnuclear cell extract loaded on 8.5% SDS-PAGE. Resolved proteins were transferred to membrane overnight, blots incubated with a blocking solution for 1 h, incubated with appropriate primary and secondary antibodies and the signals detected by ECL.

9. Measurement of Ras Activation

Cells (10×10⁶) were washed and incubated with 0.8 mCi/ml ³²Pi for 4 h in phosphate-free DMEM. Cells treated with C₂-ceramide were lysed in 500 μl lysis buffer consisting of 25 mM Tris (pH 7.5), 1% Triton X-100, 150 mM NaCl, 20 mM MgCl₂, 0.2 % SDS, 0.5% sodium deoxycholate, and 10 μg/ml of aprotinin and leupeptin. Cell lysate was immunoprecipitated with monoclonal anti-Ras and bound nucleotides eluted with 1 mM EDTA/25 μM GDP and GTP were analyzed by TLC using 0.75 M KH₂PO₄ (pH 3.5). After autoradiography, the radioactive spots corresponding to GDP and GTP were counted.

10. Cell Growth Analysis

Cell growth was determined by counting viable cells using a hemocytometer. Cells were seeded at 2.5×10⁴ per well in 6 well plates and cultured in a regular media. After the indicated period of time, cells were trypsinized and cell suspension counted for viable cells using trypan blue stain. In parallel experiments, cell growth was estimated by MTT assay. Cells were plated at 5×10³ per well in a 24 well plate, cultured in a regular media and MTT assay performed as described above in “cell viability assay” section.

11. Statistical Analysis

Statistical analyses for the comparison of control cells vs. PPT1 cells were performed by Student's t-test and results were considered statistically significant when p<0.05.

B. pH Profile of PPT1 Activity in Parental and Transfected LA-N-5

When LA-N-5 human neuroblastoma cell line was assayed for PPT1, it showed a moderate level of endogenous PPT1 activity with optimum activity at pH 4.0. In order to investigate the role of PPT1 in neuronal cells, the human PPT1 gene was prepared by RT-PCR, cloned it in a mammalian expression vector, pcDNA 3.1, and transfected into LA-N-5 cells. After selection for 3 weeks in the presence of antibiotics, stably transfected clones were isolated and tested for PPT1 activity. Enzyme activity was increased 2-3 fold in a PPT1-overexpressing cell, with a pH 4.0 optimum. The enzyme activity was phenylmethylsulfonyl fluoride (PMSF)-insensitive, as previously shown (Camp and Hofmann, 1993). Thus, preincubation with 1 mM PMSF did not affect the enzyme activity in either control or PPT1 transfected cells.

In order to verify transfection and expression of PPT1 gene, Northern blot analysis was performed, which showed a greatly increased signal when probed with [³²P] PPT1 in transfected cells. In vitro translation of cloned PPT1 was done to verify the identity of the protein. Analysis of the transcription/translation reaction product by SDS-PAGE showed a single protein band with a molecular weight of 34 kDa, which is consistent with the size predicted from the full length cDNA. To address the biosynthesis and post-translational modification of exogenously expressed PPT1, full-length PPT1 was inserted into a pFLAG expression vector to be tagged with the FLAG epitope at the C-terminal end, and transfected into LA-N-5 cells. Western analysis of transfected cell extract revealed a major protein band with a molecular weight of around 39 kDa. PPT1 has been previously shown to possess several sites for N-glycosylation, and the finding of a shift in the apparent molecular weight, consistent with those found in COS and Sf9 cells (Camp et al., 1994), indicates the similar bio-processing and modification of recombinant proteins in these cells.

As an attempt to establish a model for PPT deficiency, overexpression of dominant negative gene (inactive gene) strategy was employed. A point mutation prevalent in Finnish INCL patients (A to T mutation at nucleotide position 364 of PPT1), which is known to produce protein lacking functional enzyme activity (Vesa et al., 1995), was introduced by site-directed mutagenesis. Mutant PPT1_{A→T} construct ligated into pcDNA 3.1 was transfected and individual clones selected as described above. However, when PPT activity was measured in these cells, none of the clones showed attenuation of the endogenous PPT activity, and thus was not further investigated. Interestingly, the same mutant construct was unable to block the basal PPT1 activity in transiently transfected COS-1 cells (Vesa et al., 1995).

C. Induction of Apoptosis by C₂ Ceramide and LY-294002 in LA-N-5

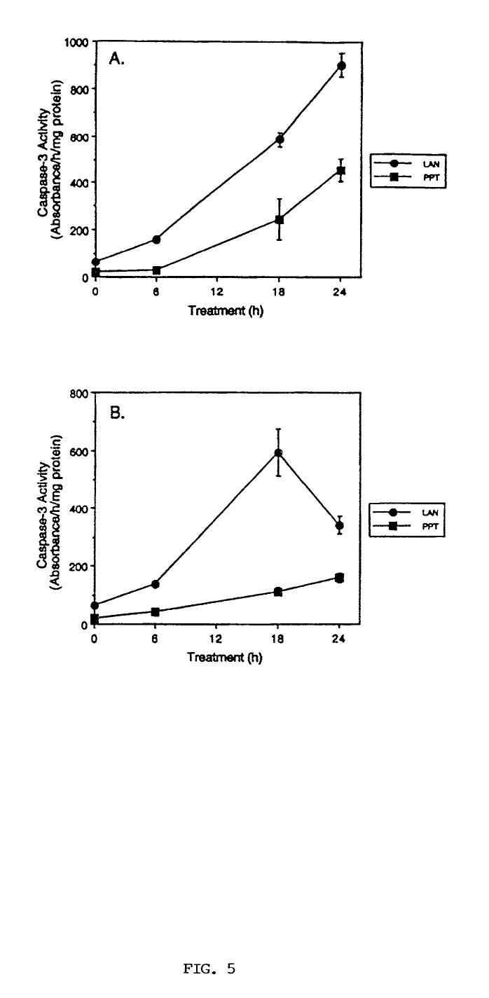

Based on the early and selective loss of neurons in the cortical area of INCL brain, the possibility of involvement of PPT1 in apoptosis in the neuronal cell line was explored. It has been established that treatment with P13-kinase inhibitors or a membrane-permeable form of ceramide such as C₂-ceramide causes cell death by apoptosis (Wiesner and Dawson, 1996; Dawson et al., 1997; Obeid et al., 1993; Haimovitz-Friedman. 1997), confirmed by DNA fragmentation, loss of cell viability and DNA ladder formation. In LA-N-5 cells, the P13-kinase inhibitor, LY-294002 (30 μM) and C₂ ceramide (25 μM) induced about 50% cell death in 24 hrs as determined by MTT assay (FIG. 4A). The drug-induced killing of LA-N-5 cells was accompanied by an increase in DNA fragmentation (FIG. 4C), one of the hallmarks for apoptotic cell death, as a result of degradation of chromosomal DNA. Furthermore, the same treatment of LA-N-5 with these drugs induced time-dependent activation of caspase 3 (FIG. 4B). It is known that pro-caspases undergo catalytic cleavage upon receiving apoptotic signals to generate shorter fragments, which are the enzymatically active form (Han et al., 1997; Yang et al., 1998). Consistent with the increased enzyme activity following drug-treatments, measured using DEVD-AFC as the substrate, Western blot analysis of LA-N-5 cells probed with the antibody against caspase 3 showed an increased level of the active 17 kDa subunit of caspase 3 following treatment with either LY-294002 or C₂-ceramide (FIG. 5C). Caspase-mediated apoptosis signaling by these drugs is in line with other studies, where preincubation with caspase inhibitors prevented cell death in Jurkat cells and sympathetic neurons (Crowder and Freeman, 1998; Mizushima et al., 1996).

D. Overexpression of PPT1 Attenuates Drug-Induced Activation of Caspase 3 and Apoptosis

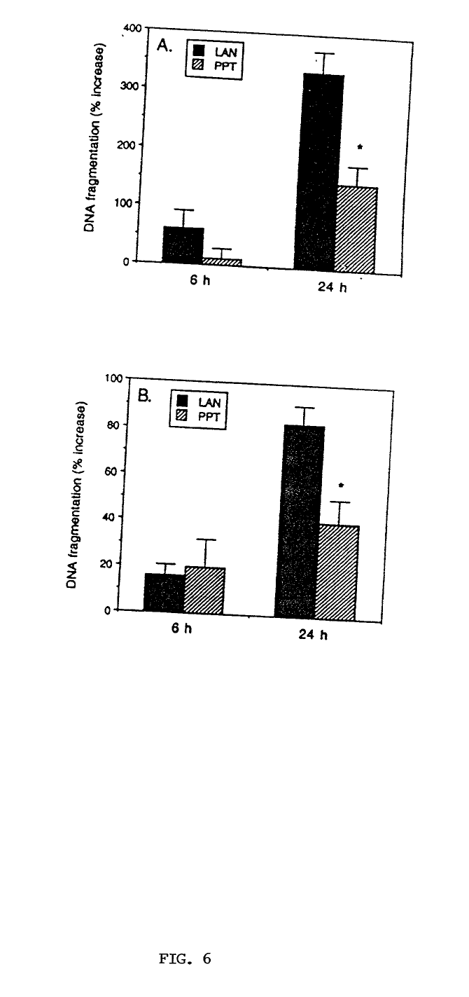

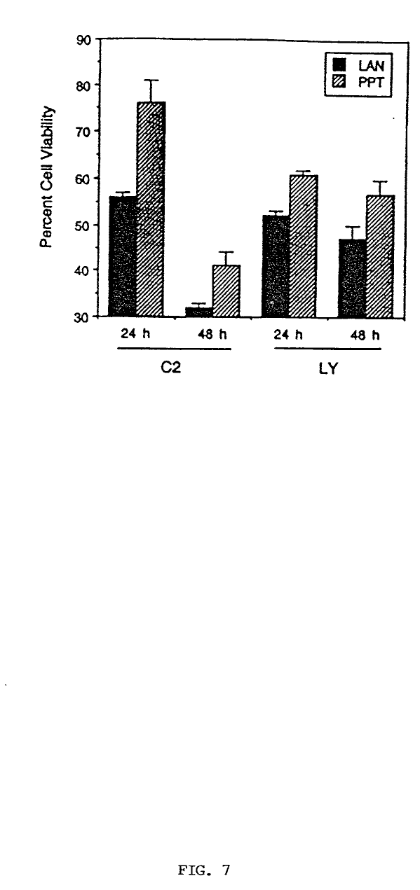

Vector control or PPT1-overexpressing LA-N-5 cells were compared for their susceptibility to drug-induced apoptosis. When caspase 3-like activity was assayed, the basal level of caspase activity was lower in PPT1-transfected cells compared to vector-transfected cells (FIG. 5). After 24 h of treatment, activation of caspase 3 in PPT1-transfected cells by C₂ ceramide or LY294002 was only 47-50% of that in control cell line (FIGS. 5A, B). Protection against activation of caspase 3 following these drug treatments was also observed by immunoblotting, showing a significantly lower level of the active 17 KDa subunit in PPT cells (FIG. 5C). The increase in DNA fragmentation following treatment with these drugs was significantly reduced in cells overexpressing PPT1, in both cell cytosol and the culture medium (FIG. 6). Cell viability measured by the MTT assay also showed fewer cells dying through apoptosis in PPT1 cells (FIG. 7). The protection against drug-induced apoptosis by PPT1 may be neuron-specific since overexpression of PPT1 in COS-7 cells was unable to attenuate either the activation of caspase 3 or DNA fragmentation following the same drug treatment.

E. Exogenous C₂ Ceramide Activates Ras.

p21^{Ras} is a key regulator in the signaling pathways involved in variety of events such as cell proliferation, transcription and apoptosis. Whether ceramide-induced apoptosis in LA-N-5 cells involves Ras activation was investigated. It is known that Ras is activated by recruitment to the membrane subdomain through palmitoylation (Cadwallader et al., 1994; Dudler and Gelb, 1996). Therefore, the subcellular localization of Ras protein was analyzed after C₂-ceramide treatment. Cells were treated with C₂ ceramide (30 μM), harvested at 10 min and 60 min and the membrane fraction (100,000× g pellet) analyzed by immunoblotting. Treatment with C₂ ceramide induced a time-dependent increase in membrane-association of Ras. Further, a Ras-guanine nucleotide binding assay showed an increase in GTP binding to Ras following treatment with C₂ ceramide. The GTP/(GDP+GTP) ratio increase over control was 33% after 10 min and 85% after 60 min treatment. These results confirmed the ceramide-mediated activation of Ras (Zhang et al., 1997; Gulbins et al., 1995).

F. PPT1 Overexpression Reduces the Level of Membrane-Associated Proteins and Decreases Cell Growth

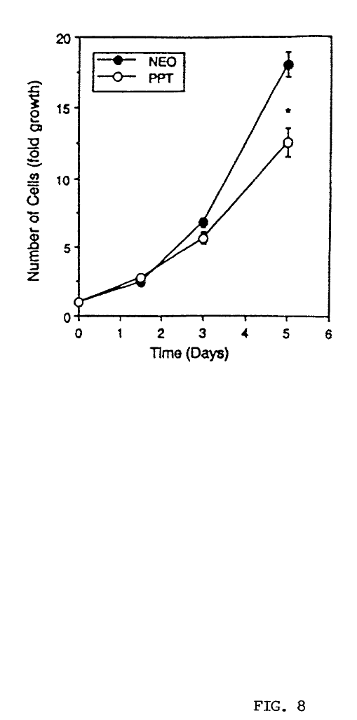

In an attempt to elucidate the mechanism of protection from apoptosis by overexpression of PPT1, the overexpression of PPT1 was hypothesized to result in a decrease in the level of palmitoylation of proteins involved in cell growth/cell cycle regulation or in the apoptotic pathway itself. PPT1 overexpression reduced membrane-associated Ras by 30-50% and reduced translocation of growth-associated protein 43 (GAP-43) from cytosol to membrane by 30%. Consistent with decreased Ras activation, cell growth assay demonstrated a slower growth rate of PPT1 overexpressing cells. When same number of cells were seeded and grown in DMEM with 10% serum, PPT1 cells were reduced to 70% of control cells following 5 days of incubation (FIG. 8). This result was confirmed by MTT assay.

G. Increased Resistance to Apoptosis in PPT1 Cells Is Associated with Increased Akt Activation.

To further investigate the downstream pathway by which PPT1 exerts protection against apoptosis, the activation of Akt was examined in control and PPT1 overexpressing cells. Akt is an anti-apoptotic serine/threonine protein kinase (Zhou et al., 1998), which itself is activated upon phosphorylation of Ser473 and Thr308 residues (Alessi et al., 1996). Therefore, the activation of Akt was analyzed by immunoblotting the cell extracts with an antibody specific to phosphorylated Akt (Ser473). As a positive control for the activation of Akt kinase and its phosphorylation, cells were stimulated with a growth factor such as insulin. Treatment of control vector-transfected cells with insulin for 5 min induced significant phosphorylation of Akt, consistent with previous reports (Zhou et al., 1998, Alessi et al., 1996). Pretreatment with a P13 kinase inhibitor, LY294002, blocked the insulin-induced effect, confirming the P13 kinase-dependent nature of Akt activation. Phosphorylation of Akt was also demonstrated by the appearance of a slow migrating band (higher molecular weight) shown in Akt 2 immunoblot. Interestingly, PPT1-overexpressing cells exhibited a greatly increased level of basal as well as insulin-mediated Akt phosphorylation compared to vector-transfected cells, which is in agreement with the decreased apoptosis found in PPT overexpressing cells. Immunoblotting for total Akt proteins (Aktl/Akt2) showed a similar level of protein expression under all conditions, suggesting the involvement of a post-translational modification rather than gene expression.

### Example 3

### PPT1 Modulator Treatment Inhibits PPT1 Activity and Increases Cell Death

A. Materials and Methods

1. Materials

C₂-ceramide was purchased from Matreya Inc. (Pleasant Gap, Pa.), MTT, pFLAG-CMV-5a, FLAG-M2 antibody and anti-mouse FITC conjugate from Sigma (St. Louis, Mo.), propidium iodide from Oncor (Gaithersburg, Md.), and Tfx-50 transfection reagent and pSV-p-galactosidase from Promega (Madison, Wis.).

2. Construction of Sense and Antisense PPT1 Expression Vectors

cDNA for the human PPT1 gene was prepared by reverse-transcription polymerase chain reaction using a set of primers with appropriate restriction enzyme sites (Cho and Dawson, 2000; GenBank Accession number L42809). The amplified sequences of DNA had KpnI (5′ end)/XbaI (3′ end) insertion sites for sense DNA and XbaI (5′ end)/KpnI (3′ end) sites for antisense reversed DNA. Each of the amplified genes was inserted into the multiple cloning site of pcDNA 3.1 to generate either sense PPT1 or antisense PPT1. The orientation of each construct was verified by sequence analysis and used for transfection of cells. FLAG-tagged PPT1 was prepared by inserting PPT1 into a C-terminal pFLAG-CMV-5a expression vector.

3. Cell Culture and Transfection

LA-N-5 human neuroblastoma cells were grown in DMEM supplemented with 10% fetal bovine serum and 1% gentamycin. Transfection was performed using Tfx-50 transfection reagent according to the manufacturer's instructions. At 48 h post-transfection, cells were harvested or treated appropriately depending on the experimental procedure. Clones of cells stably expressing PPT1 were selected and maintained by growing in media containing G418 (500 μg/ml) (as described above).

4. PPT1 Assay

PPT1 activity was measured as described above. In brief, the cell sonicate was incubated in the assay mixture (50 mM sodium citrate (pH 4.0) for [¹⁴C]palmitoylated IRYCWLRR Po peptide substrate (3,000-4,000 cpm) or 50 mM Tris (pH 7.4) for [¹⁴C]palmitoylated MLCCMRR GAP43 peptide substrate) for 20 min at 37° C. After organic extraction, samples were analyzed by HPTLC using n-butanol/pyridine/acetic acid/water (45:30:9:36, by volume) and the radioactive spot corresponding to [¹⁴C] palmitate was counted.

5. Western Blot Analysis

Cells were harvested in a lysis buffer containing 10 mM Tris (pH 7.4), 1 mM EGTA, 1 mM DTT, and 10 μg/ml aprotinin, leupeptin and pepstatin. After lysis on ice for 1 h, cell debris was removed by centrifugation at 3,000 rpm for 5 min and equal amounts of post-nuclear cell extract were resolved by SDS-PAGE. Resolved proteins were transferred to membrane overnight, blots incubated with a blocking solution for 1 h, incubated with appropriate primary and secondary antibodies and the signals detected by enhanced chemiluminescence.

6. Immunocytochemistry

Cells were prepared on glass chamber slides precoated with poly-lysine. At 48 h post-transfection, cells were washed with Tris-buffered saline containing 0.05% Tween 20 and 1 mM calcium chloride (TBST/Ca) and fixed with an acetone:methanol mixture (1:1) for 1 min. After rinsing with TBST/Ca, cells were blocked with 2% BSA and incubated with FLAG-M2 antibody (10 μg/ml) for 1 h. Nonspecific binding was washed away with TBST/Ca and the binding of secondary antibody conjugate to FITC was analyzed with a Zeiss Axiovert S100 TV microscope equipped with an epifluorescent unit, a ZVS-3C7DE three chip video camera and KS300 3.0 imaging software (Imaging Core facility, Department of Pediatrics, University of Chicago).

7. Propidium Iodide Staining

Cells on chamber slides were transfected as described above. At 48 h post-transfection, cells were treated with 15 μM C₂ ceramide for 6 h to initiate apoptosis, washed with serum-containing media and PBS twice each, and incubated with PI for 10 min according to the manufacturer's protocol. The binding of PI was visualized by fluorescent microscopy as described above using a rhodamine filter.

B. Overexpression of Epitope-Tagged PPT1 Is Consistent with a Lysosomal Hydrolase

In order to show the overexpression and intracellular localization of transfected PPT1 in LA-N-5, sub-confluent cells were transiently transfected with FLAG-tagged PPT1. After 48 h, the protein expression was visualized by immunocytochemistry using anti-FLAG-M2 antibody, followed by incubation with FITC-conjugated secondary antibody. Fluorescent microscopic observation of transfected cells revealed a punctate pattern of strong staining signals throughout the cytoplasmic compartment, consistent with a previous finding (Salonen et al, 1998), suggesting the primary lysosomal localization of PPT1. Expression of recombinant PPT1 in these cells was also confirmed by a specific immuno-interaction of FLAG- M2 antibody with an approximately 38 kDa PPT1 protein by Western blot analysis.

C. PPT1-FLAG Expression is Inhibited by Co-Transfection of Reverse-Sequence PPT1

Establishment of a cellular model in which PPT1 activity is compromised is essential to study the pathophysiological consequences of PPT1 deficiency in INCL. An initial attempt was made to generate a neuronal cell line by knocking-out PPT1 protein by stably transfecting antisense PPT1 (AS-PPT1). The initial period of clonal selection under neomycin pressure resulted in the death of >80% of the cells as expected, probably representing the death of untransfected cells. However, after 3 further weeks of selection, PPT1 enzyme activity was still detectable at normal levels in the surviving cells. This was repeated 3 times with the same result.

In order to examine whether the addition of AS-PPT1 could block the synthesis of PPT1, LAN-5 cells were then transiently transfected with either PPT1-FLAG alone (1 or 2 μg) or in combination with AS-PPT1 (3 μg). After 48 h, cells were harvested and SDS-PAGE-resolved protein extracts were immunoblotted with anti-FLAG antibody to analyze the expression of FLAG-tagged PPT1. A dose-dependent increase in the expression of PPT1 was observed in transfected cells and when the same amount of PPT1 was co-transfected with AS-PPT1, the expression of PPT1 was reduced to an undetectable level. Only very small amounts of PPT1 were detectable in AS-PPT treated cells when the film was overexposed suggesting that the inhibition of synthesis was >95%. Thus the antisense sequence was effective in blocking PPT1 synthesis and the failure to generate stable clones of PPT-negative neuronal cells suggests that PPT1 may be essential for survival.

The inhibition of PPT1 expression by AS-PPT1 was also confirmed by immunofluorescence analysis. Cells on chamber slides were transfected with PPT1-FLAG with or without AS-PPT1. After 48 h, cells were fixed and immunostained slides were observed under fluorescent microscopy. Addition of PPT1 resulted in a robust expression of FLAG epitope-tagged protein. In contrast, co-transfection of AS-PPT1 with PPT1-FLAG efficiently inhibited expression of PPT1. The inhibition by AS-PPT1 was specific since addition of a non-PPT1 sequence, e.g., that for β-galactosidase, did not induce an inhibition of PPT1 expression.

D. Lowering PPT1 Activity by AS-PPT1 Transfection of Cells Increases Cell Death Induced by C₂-Ceramide

Whether inhibition of PPT1 by AS-PPT1 treatment would result in increased apoptosis was investigated. LAN-5 cells were transfected with either PPT1 or AS-PPT1 and after 48 h, they were treated with 15 μM C₂-ceramide for 6 h to induce apoptosis (Weisner & Dawson, 1996a,b; Goswami & Dawson, 2000). Cells were rinsed with serum-containing media, incubated immediately with propidium iodide (PI) for 10 min and observed by fluorescent microscopy. AS-PPT1 treated cells had a significantly increased number of PI-positive cells (indicative of increased apoptosis) compared to PPT1-transfected cells.

E. PPT1 Antisense Treatment Reduces PPT1 Activity

To provide further evidence that treatment with AS-PPT results in inhibition of PPT1, PPT1 enzyme activity was measured directly by an in vitro assay (Cho & Dawson, 1998), using cell extracts from either control or AS-PPT1- treated cells. Addition of AS-PPT1 resulted in a 12%-18% reduction of PPT1 activity compared to control, when either [¹⁴C]-palmitoyl-IRYCWLRR (Po) or [¹⁴C]- palmitoyl-MLCCMRR (GAP43) peptide were used as the substrate (FIG. 9). The reduction in PPT1 activity compares well to the expected cell transient transfection efficiency of 10-20%.

F. Treatment with a PPT1 Inhibitor Increases Cell Death

In order to further verify that the increased susceptibility to drug-induced apoptosis of the AS-PPT1 treated cells was due to compromised PPT1 activity, the extent of cell death induced by C₂-ceramide treatment in control was compared to DAP 1-treated cells. Treatment of cells with DAP1 alone induced 5-10% death of cells at DAP1 concentrations up to 100 μM for 6 h., as measured by MTT assay (FIG. 10A). Incubation of cells with 30 μM C₂-ceramide alone for 6 h also resulted in about 10% death of cells, but preincubation with DAP 1 (up to 100 μM) for 1 h prior to the addition of C₂-ceramide (30 μM) increased the cell death by almost 3-fold to 28% (FIG. 10A). In order to further prove that the increased cell death induced by C₂-ceramide treatment was the consequence of PPT inhibition, the effect of the same treatment in cells where we had overexpressed PPT1 by more than 2-fold (Example 1) was compared. In these cells, neither C₂-ceramide, DAP1 alone or DAP1 (100 μM) followed by C₂-ceramide (30 μM) for 6 h induced more than 10% cell death (FIG. 10B).

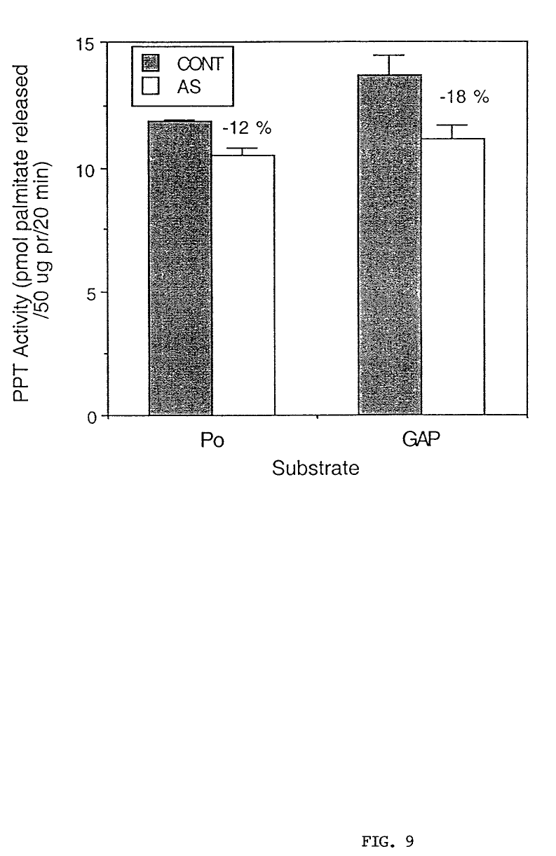

The effect of DAP1 on etoposide-induced cell death was examined and its was found that treatment with DAP1 (100 mM) alone for 24 h produced 20-25% cell death and that co-treatment with etoposide and DAP1 (100 μM) for 24 h increased cell death from 40% to 60% over the concentration range 0-50 μM etoposide (FIG. 11A). Thus the co-addition of DAP1 gave the same percent cell killing as 100 μM etoposide, (FIG. 11B), suggesting that DAP1 can reduce the amount of the chemotherapeutic drug required to kill cells by 50%.

G. DAP1 Has Anti-Growth Activity Against a Variety of Cancer Cells

Preliminary data from an NCI screen suggests that the compound DAP1 by itself has anti-growth activity (at least 50% inhibition of growth and in some cases 100% inhbition of growth; concentrations used were in the range 10-100 μM) against the following cell lines in Table 6:

TABLE 6
Cancers and Cancer Cell Lines

| | |
|---|---|
| leukemia | CCRF-CEM |
| Non-small cell lung cancer | HOP-92 |
| | NCIH332M |
| Colon Cancer | HCT-15 |
| Melanoma | MALME-3M |
| CNS Cancer | U251 |
| | SNB-19 |
| Ovarian cancer | IGROV1 |
| | OVCAR-3,4 and 8 |
| Renal cancer | 498 |
| | 786-0 |
| prostate cancer | DU-145 |
| | PC-3 |
| Breast cancer | T47D |
| | MDA-MB-231/ATCC |

Over 60 differed strains of cell lines were tested and growth rate was reduced in all of them compated to control. Given its efficacy against a wide number of cancer cell types, DAP 1 and related compounds should be effective in reducing the growth of many types of cancer. However, the range of effect on the inhibition of growth rates suggests that DAP1 could be the first generation of a novel class of antitumor drugs showing tissue type specificity. (Modified

From page 7 of the grant proposal under PPT1 as a potential anti-cancer drug target “The amide had much less effect on COS cells and embryonic neuron primary cultures, suggesting that it could be the first generation of a novel class of antitumor drugs with some degree of specificity”)

### Example 4

### Reduction of Tumor Growth in vivo

A. Materials and Methods

1. Materials

A tumor cell line, such as LAN-5, a human neuroblastoma cell line will be employed. Such cell lines may be obtained through ATCC (www.atcc.org). Tumor cells will be injected into nude mice. PPT1 inhibitors will be synthesized or purchased. In addition the mice may be given a chemotherapeutic agents, such as etoposide and daunorubicin.

2. Methods of Assaying for Tumor Growth

Initially, the human neuroblastoma cell line LAN-5 dissociated cells (4×10⁷) will be injected subcutaneously into nude mice. Animals will be monitored for tumor growth (e.g., Nakagawa et al., 2000) at 11, 22 and 33 days and at time of death. Once optimum doses are determined a thorough anatomical analysis will be conducted to identify micrometastases and any evidence of abnormal pathology. All studies will be carried out under NCI guidelines and the recommendations of the American Veterinary Association.

B. Chemotherapeutics in Combination with a PPT1 Inhibitor

Etoposide and daunorubicin IV will be administered to the mice bearing tumors. Tumor growth will be monitored by weighing the tumor and checking adjacent tissue for metastases as described by Wang et al., 2000. Drug concentrations that will used should be sufficient to reduce tumor size by 50%. Parallel set of experiments will be conducted in which DAP1 and related compounds are added (0.001-lOg/kg) to determine concentration ranges that effect 100% tumor reduction. The absence of metastases will be evaluated and the life-span of such animals will be evaluated. Animals will be treated with DAP1 alone at increasing concentrations to establish toxicity. Once optimum doses are determined thorough anatomical analyses will be conducted to identify any micrometastases and any evidence of abnormal pathology.

C. DAP1 Modifications

Shortening the fatty acid part of DAP1 from C16 to C12 or C8 reduced the potency slightly but greatly improved the solubility. Removing VKIKK greatly increased the hydrophobicity of the peptide. Its potency as a PPT inhibitor will be evaluated in an in vitro assay.

The alpha-keto-amide form of DAP1 has been created using the Dess Martin oxidation. Starting with alpha hydroxy-octanoic acid, alpha hydroxy-DAP1 have been made and reduced to the alpha-keto-DAPI.

Keto amide-based inhibitors of proteases have been synthesized by others to take advantage of interactions between the S1′ and S2′ positions of substrates (C-terminal of the cleavage site) with the P1′ and P2′ pockets of the enzyme, in addition to the more commonly utilized P1, P2 and P3 binding pockets (N-terminal of the cleavage site). These inhibitors have been shown to have greater specificity than traditional fluorinated ketone-type inhibitors in these systems (Ogilvey et al, 1997; Slee et al., 2000) Recently, c-keto amide triglyceride analogs have been synthesized as inhibitors of Staphylococcus hyicus lipase (Simmons et al., 1995) and pancreatic lipase (Chiou et al., 2000). Although the mechanism of action of α-keto amides is not well established, inhibitors based on this group have been used successfully for a variety of hydrolytic enzymes including esterases, serine proteases and aspartyl proteases such as the HIV protease.

PPT1 inhibitors incorporating the α-keto amide function should therefore have greater potency and will be designed using the structural information derived from our studies of substrate specificity and DAP1 analogs described above. Since all inhibitors will be tested immediately in the rapid, fluoresecent (4MU-based) PPT1 assay, this will allow for direct feedback into the inhibitor design process. Synthesis of these inhibitors will be performed by both solution and solid phase approaches.

Alphahydroxyhexadecanoic acid is not commercially available and will be custom-synthesized. The C16-keto-amide DAP1 is expected to be a potent inhibitor of PPT and a potent anti-cancer compound.

### Example 5

### PPT Activity in Cultured Cells

PPT activity was determined by measuring the hydrolysis of 4-methylumbelliferyl-beta-D glucosyl 6-thiopalmitate. PPTs hydrolyze the 6-thioplamitate and commercial almond beta glucosidase hydrolyzes the glucose to release the 4MU. The fluorescent intensity of 4MU is a measure of enzyme activity. Extracts of cultured human skin fibroblasts were assayed; patients with the infantile form of Battens disease expectedly had no enzyme activity. Normal cells had an activity range of 50-100 units. Lymphoblast lines from human patients had activity in the range 25-40. In contrast when extracts from cells grown from human malignant tumors (neuroblastomas, gliomas, oligodendrogliomas, etc.) were assayed, the activity was in the range 1000-2000 units. This suggests that tumor cells may have a very active PPT, which protects them against the body's cell killing mechanisms, allowing the tumor cells to grow rapidly and metastasize.

### Example 6

### Role of PPT1 in Cell Death

A. Materials and Methods

C2-Ceramide was purchased from Matreya Inc. (Pleasant Gap, Pa.), MTT from Sigma (St. Louis, Mo.), and synthetic peptides from Research Genetics (Huntsville, Ala.). DAPl was synthesized as described in a previous publication (Cho et al., 2000a).

LA-N-5 human neuroblastoma cells were grown in DMEM supplemented with 10% fetal bovine serum and 1% gentamicin. Clones of cells stably expressing PPT1 were selected and maintained by growing in media containing G418 (500 μg/ml) as described (Cho, et al., 2000b). Cell death was measured by the MTT method (Cho et al., 2000a; Cho et al., 2000b; Cho et al., 2000c).

B. PPT1 and DAP1 Effects on Activity of Pro-Apoptotic Drugs

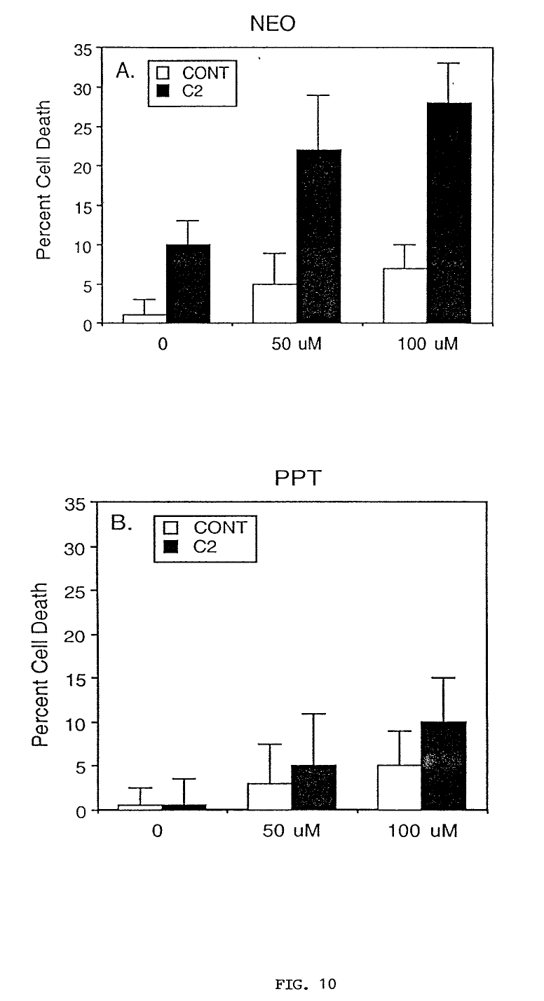

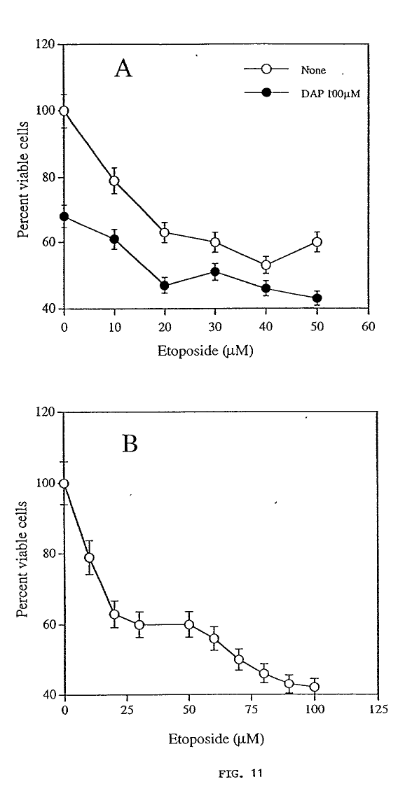

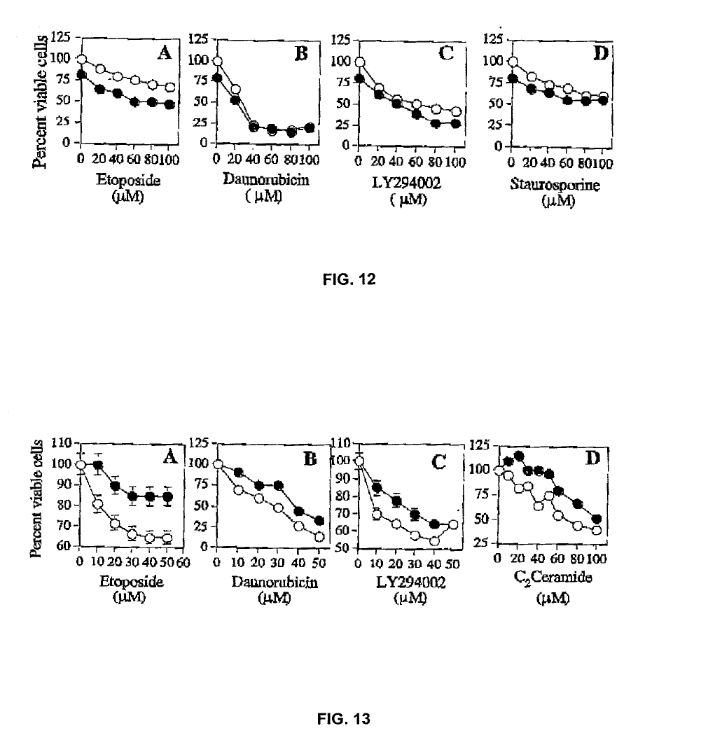

When DAP1 was added to LAN-5 cultured cells, after 18 hours up to 20% cell death was observed, and the drug increased killing by the following commonly used, DNA-targetting drugs: etoposide, daunorubicin, P13 kinase inhibitor LY294002, and C2-ceramide (FIGS. 12A-D). Presumably this occurred by different mechanisms.

LAN-5 cells were compared to LAN-5 cells transfected with PPT1 to determine whether the reverse effect could ve observed-that increased PPT1 protects against cell death. The cells overexpressing PPT1 were demonstrated to have a markedly decreased susceptibility to killing by agents that induce apoptosis-etoposide, daunorubicin, LY924002, and staurosporine (FIGS. 13A-D).

### Example 7

### Synthesis of a Ketoamide Form of DAP1 and its Potency

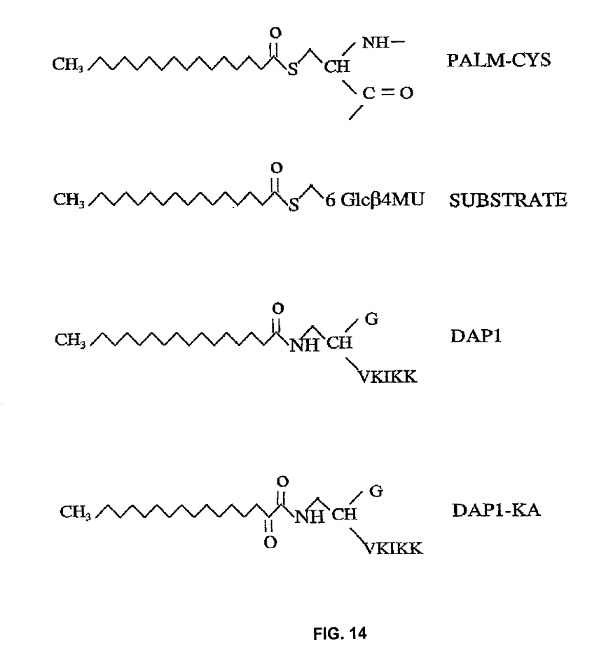

Keto amide- and keto ester-based unhibitors of proteases have been shown to have greater specificity than traditional fluorinated ketone-type inhibitors in these systems (Ogilvie et al., 1997; Slee et al., 1995). Recently, α-keto amide triglyceride analogs have been synthesized as inhibitors of Staphylococcus hyicus lipase (Simmons et al., 1999) and pancreatic lipase (Chiou et al., 2000). The fully protected peptide AcG-Dap((+/−)α-hydroxyhexadecanoyl)-VKIKK.amide (DAP-KA) was synthesized by solid phase peptide synthesis in good (mg) yield (FIG. 14).

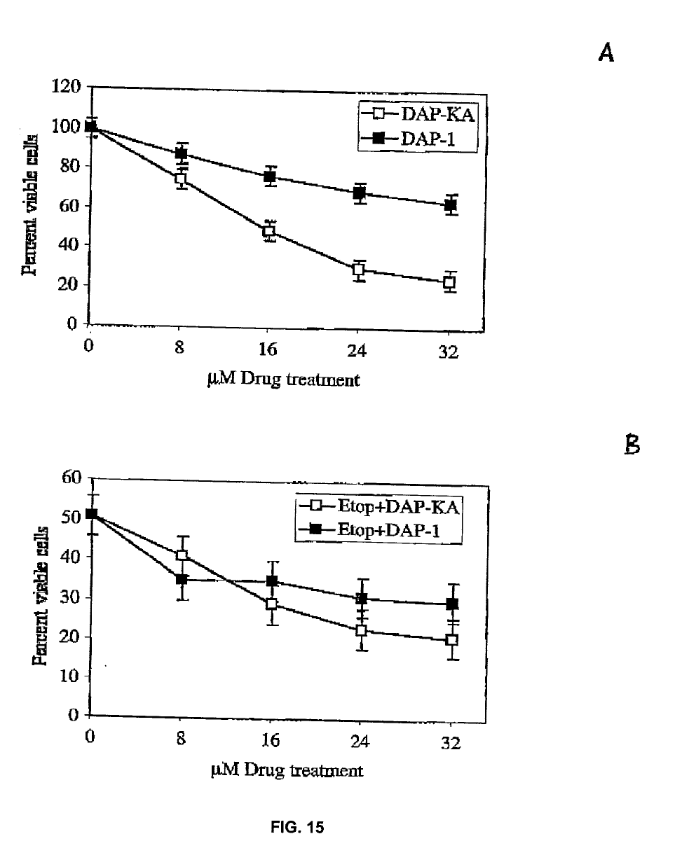

The relative potency of DAP1 compared to DAP1 ketoamide (DAP-KA) in killing cells was evaluated. HOG cells, human oligodendroglioma cells, were exposed for 24 hours to a) DAP1 or DAP-KA (FIG. 15A) or 10 mM etoposide and either DAP 1 or DAP-KA (FIG. 15B). The concentration of etoposide was chosen to give 50% killing as measured by MTT assay. Apoptotic cell death was confirmed by demonstrating DNA fragmentation. DAP-KA proved to be a more potent inhibitor than DAP1 and it was shown to be a more potent inducer of cell death than DAP1 by enhancing the killing of human oligodendroglioma cells by etoposide.

### Example 8

### Modification of the Peptide Moiety

A. Non-Peptide Inhibitors

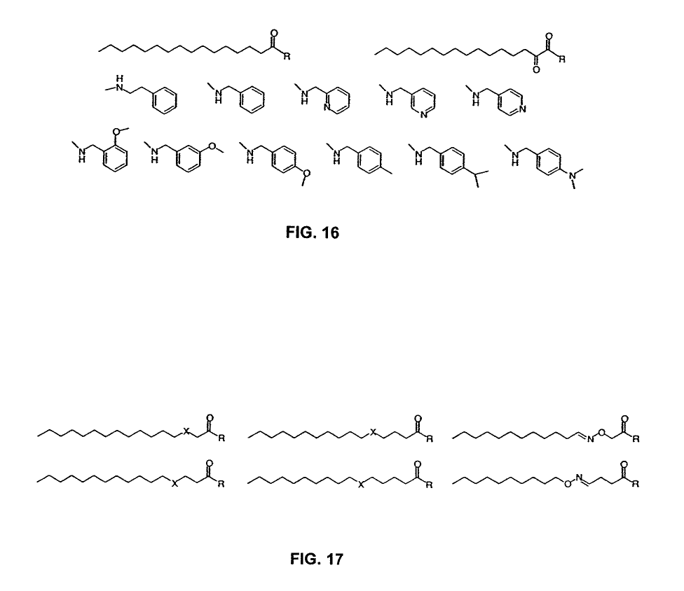

The peptidyl component of inhibitors described herein will be eliminated by synthesizing non hydrolyzable amide inhibitors between the following amines, and palmitate or other lipid analogs as in FIG. 16. The proposed binding pocket in the PPT1:palmitate structure (1EH5.pdb) has a few potential hydrogen bonding interactions but is largely hydrophobic, including the aromatic face of Phe114. A series of substituted benzyl amines may take advantage of possible π-stacking interactions between the rings and also make hydrogen bonding contacts with the protein. If the affinity of simple amide inhibitors is not sufficient, it will be straightforward to synthesize the corresponding α-ketoamide inhibitors.

B. Modification of the Lipid Moiety

Initial synthetic studies done by the inventors showed that decreasing the fatty acid chain length (C14, C12, C8) reduces the efficacy of the inhibitor, so lipid modifications shown in FIG. 17 will be implemented.

Such modifications will specifically include the N═C double bond change shown above. The double bond causes the structure to kink and thus fit better into the palmitoyl-PPT1 cyrstal structure active site. The oxime ether shown above will also be implemented in some molecules. An interesting observation from the palmitoyl-PPT1 crystal structure (1EH5.pdb) is that the entire palmitoyl group is bound in a tight hydrophobic groove, that exposes the even numbered methylene groups to solvent. More importantly, the inventors have observed a remarkable fit of this region with the sphingosine base structure (Huwiler et al., 2000), which will be that basis for future modifications. Palmitate derivatives with a Δ⁴ or Δ⁵ double bond will be synthesized using Wittig chemistry, using 4-bromo-proprionic acid and dodecanal as starting materials.

Alternatively, introduction of heteroatoms (X=O,S) into the lipid backbone in this region might accommodate this bent structure. Ether and thioether analogs shown inf FIG. 17 can be readily synthesized from substituted longchain hydrocarbons. An additional approach is to incorporate an oxime ether into the lipid, positioning the double bond at the C4-C5 position (C5-C6 analogs could also be synthesized).

C. α-Keto Heterocycle Inhibitors

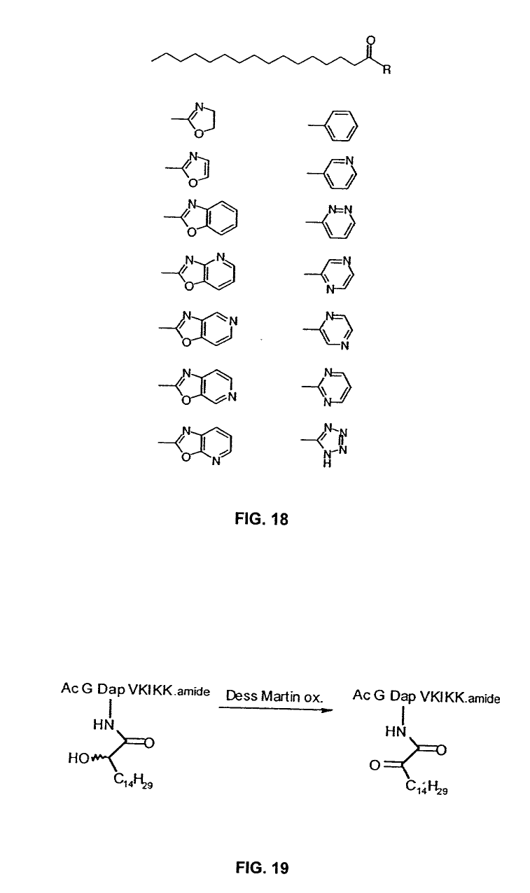

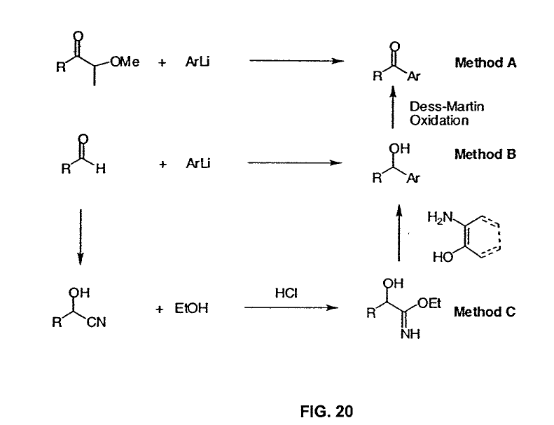

A number of enzyme inhibitors have been described based on α-keto heterocycles, such as a proposed palmitoylated example shown in FIG. 18, including serine protease inhibitors. Like ketoamide inhibitors, this class of inhibitors combine an electrophilic carbonyl with additional moieties that may contribute independently to the binding affinity. In addition to serine proteases such as chymotrypsin, OleoylΔ^{9,10}-α-Keto heterocycles have been shown to be potent inhibitors of FAAH (fatty acid amide hydrolase) a serine hydrolase that cleaves unsaturated lipid amides such as oleamide.

The oxazole and benzoxazole groups and their derivatives have been shown to be exceptionally potent for both chymotrypsin and FAAH (in comparison to the analogous benzthiazole and benzimidazole derivatives) so the inhibitors shown in FIG. 18 will be created. Future studies will focus on substitutions to the benzoxazole ring. Nitrogen substituents on the six membered rings may be able to take advantage of the water mediated hydrogen bonding to the exposed backbone carbonyls present in the proposed peptide binding pocket observed in the crystal structure of the palmitoyl-PPT1 complex. This class of inhibitors will also be compatible with any lipid analog described herein.

D. Methods for Synthesis of Keto Amides

An inhibitor using the α-keto amide moiety (described for DAP-1 ketoamide) can be synthesized by coupling a C16:0 α-hydroxy fatty acid (commercially available from Sigma) instead of palmitate described in the DAP 1 synthesis. (The corresponding α-keto ester inhibitor can be synthesized analogously through coupling the α-hydroxy fatty acid to a ser residue in the same position.). Following acylation, the C16 α-hydroxy amide group was oxidized to the desired α-keto amide. This oxidation has been described using the Dess-Martin periodinane (Slee et al., 1995) and pyridinium dichromate in acetic acid (Chiou et al., 2000). Subsequent oxidation of the a-hydroxy amide group in solution with Dess-Martin reagent in TFA/CH₂Cl₂ produced the α-keto amide analogue in 70% recovered yield. Interestingly, the reaction was clean despite the use of a filly unprotected peptide. The compatibility of unprotected lysine side chains with the Dess-Martin oxidation will greatly facilitate the handling and purification of these inhibitors.

As with the DAP1 amide inhibitors, it will be straightforward to modify both the peptide and lipid components of the inhibitor to optimize the potency and selectivity of the inhibitor. The α-keto amide inhibitors will be assayed by both cell based and in vitro assays as described herein and as is known in the art. Synthesis of these inhibitors will be performed by both solution and solid phase approaches.

E. Methods for Synthesis of a-Ketoheterocycle

The α-ketoheterocycles can be synthesized by published methods shown in FIG. 20. (Edwards et al., 1992). Addition of the heteroaryl lithium reagent to the Weinreb amide gives the desired α-ketoheterocycle directly (Method A). or indirectly from the aldehyde-proceeding through the α-hydroxy heterocycles followed by Dess-Martin oxidation via an addition of the heteroaryl lithium reagent (Method B) or alternatively by cyanohydrin formation, acid-catalyzed conversion to the imidate (HCl-EtOH, CHCl₃), and condensation with a 2-aminoalcohol, 2 aminophenol or o-amino hyroxypyridine (Method C).

F. Other Modifications

Further modifications are contemplated. The S on the PPT1 substrate shown in FIG. 14 will be replaced with a NH. Also, a double bond will be introduced between C4 and C5 of the palmitate moiety of a ketoamide. Moreover, the peptide portion of the DAP-ketoamide will be replaced with glucose or another sugar-based residue to improve solubility.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents, which are both chemically and physiologically related, may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,162,282

U.S. Pat. No. 4,196,265

U.S. Pat. No. 4,275,149

U.S. Pat. No. 4,277,437

U.S. Pat. No. 4,310,505

U.S. Pat. No. 4,366,241

U.S. Pat. No. 4,472,509

U.S. Pat. No. 4,533,254

U.S. Pat. No. 4,554,101

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,683,195

U.S. Pat. No. 4,683,202

U.S. Pat. No. 4,684,611

U.S. Pat. No. 4,690,915

U.S. Pat. No. 4,728,575

U.S. Pat. No. 4,728,578

U.S. Pat. No. 4,737,323

U.S. Pat. No. 4,800,159

U.S. Pat. No. 4,883,750

U.S. Pat. No. 4,879,236

U.S. Pat. No. 4,921,706

U.S. Pat. No. 4,938,948

U.S. Pat. No. 4,946,773

U.S. Pat. No. 4,952,500

U.S. Pat. No. 5,021,236

U.S. Pat. No. 5,196,066

U.S. Pat. No. 5,199,942

U.S. Pat. No. 5,279,721

U.S. Pat. No. 5,302,523

U.S. Pat. No. 5,322,783

U.S. Pat. No. 5,354,855

U.S. Pat. No. 5,384,253

U.S. Pat. No. 5,464,765

U.S. Pat. No. 5,538,877

U.S. Pat. No. 5,538,880

U.S. Pat. No. 5,550,318

U.S. Pat. No. 5,563,055

U.S. Pat. No. 5,580,859

U.S. Pat. No. 5,589,466

U.S. Pat. No. 5,591,616

U.S. Pat. No. 5,610,042

U.S. Pat. No. 5,656,610

U.S. Pat. No. 5,702,932

U.S. Pat. No. 5,736,524

U.S. Pat. No. 5,780,448

U.S. Pat. No. 5,789,215

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,486

U.S. Pat. No. 5,849,487

U.S. Pat. No. 5,849,546

U.S. Pat. No. 5,849,483

U.S. Pat. No. 5,849,497

U.S. Pat. No. 5,849,547

U.S. Pat. No. 5,851,770

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,366

U.S. Pat. No. 5,866,337

U.S. Pat. No. 5,871,986

U.S. Pat. No. 5,879,703

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,525

U.S. Pat. No. 5,925,565

U.S. Pat. No. 5,928,862

U.S. Pat. No. 5,928,869

U.S. Pat. No. 5,928,905

U.S. Pat. No. 5,928,906

U.S. Pat. No. 5,928,870

U.S. Pat. No. 5,929,227

U.S. Pat. No. 5,932,413

U.S. Pat. No. 5,932,451

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,981,274

U.S. Pat. No. 5,994,624

PCT/US85/01161

PCT/US87/00880

PCT/US89/01025

PCT/US89/05040

PCT/9506128

EP 320 308

EP 329 822

GB 2193095

GB 2202328

WO 84/03564

WO 88/10315

WO 89/06700

WO 90/07641

WO 94/09699

WO 99/18933

Abbondanzo et al., Breast Cancer Res. Treat., 16:182(#151), 1990.

Alessi et al., EMBO J., 15:6541-6551, 1996.

Allred et al., Breast Cancer Res. Treat., 16:182(#149), 1990.

Allsopp et al., Cell 73:295-307, 1993.

Almendor et al., J. Immunol., 157:5411-5421, 1996.

Angel et al., Cell, 49:729, 1987b.

Angel et al., Mol. Cell. Biol., 7:2256, 1987a.

Arap et al., Cancer Res., 55:1351-1354, 1995.

Arends and Wylie, Int. Rev. Exp. Pathol. 32:223-254, 1991.

Atchison and Perry, Cell, 46:253, 1986.

Atchison and Perry, Cell, 48:121, 1987.

Atherton et al., Biol. of Reproduction, 32, 155-171, 1985.

Ausubel, ed., Current protocols in molecular biology, New York, John Wiley & Sons, 1996.

Baichwal and Sugden, In: Gene Transfer, Kucherlapati R, ed., New York, Plenum Press, pp. 117-148, 1986.

Bajorin et al, Proc. Annu. Meet. Am. Soc. Clin. Oncol., 7:A967, 1988.

Baker, G. et al. (Eds.), Modern Phannaceutics, Marcel Dekker, Inc., New York, NY, 1990.

Bakhshi et al., Cell, 41:899-906, 1985.

Banerji et al., Cell, 27:299, 1981.

Banerji et al., Cell, 35:729, 1983.

Bangham etal., J. Mol. Biol., 13:238-252, 1965.

Bao et al., Hum. Gene Ther., 7:355-365, 1996.

Bass et al., Cancer Gene Ther., 2:97-104, 1995.

Bellus, J. Macromol. Sci. Pure Appl. Chem, A31(1): 1355-1376, 1994.

Berberian et al., Science, 261:1588-1591, 1993.

Berkhout et al., Cell, 59:273, 1989.

Berthiaume and Resh, J. Biol. Chem. 270:22399, 1995.

Bharadwaj and Bizzozero, J. Neurochem. 65:1805-1815, 1995.

Bizzozero and Good, J. Biol. Chem. 266:17092-17098, 1991.

Bizzozero, Neuropediatics 28: 23-26, 1997.

Blanar et al., EMBO J., 8:1139, 1989.

Bodine and Ley, EMBO J., 6:2997, 1987.

Boshart et al., Cell, 41:521, 1985.

Bosze et al., EMBO J., 5:1615, 1986.

Braddock et al., Cell, 58:269, 1989.

Brown et al. Breast Cancer Res. Treat., 16:192(# 191), 1990.

Brutlag et al., CABIOS, 6:237-245, 1990.

Bulla and Siddiqui, J. Virol., 62:1437, 1986.

Cadwallader et al., Mol. Cell Biol., 14:4722-4730, 1994.

Caldas et al., Nat. Genet., 8:27-32, 1994.

Camp and Hofmann, J. Biol. Chem. 268:22566-22574, 1993.

Camp et al, J. Biol. Chem. 269:23212-23219, 1994.

Campbell and Villarreal, Mol. Cell. BioL, 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campo et al., Nature, 303:77, 1983.

Canfield etal., Methods in Enzymology, 189, 418-422, 1990.

Carbonelli et al., FEMS Microbiol. Lett., 177:75-82, 1999.

Celander and Haseltine, J. Virology, 61:269, 1987.

Celander et al., J. Virology, 62:1314, 1988.

Chandler et al., Proc. Natl. Acad. Sci. USA, 94:3596-3601, 1997.

Chandler et al., Cell, 33:489, 1983.

Chang et al., Mol. Cell. Biol., 9:2153, 1989.

Chatterjee et al., Proc. Nat'l Acad. Sci. USA., 86:9114, 1989.

Chen and Okayama, Mol. Cell Biol., 7:2745-2752, 1987.

Cheng et al., Investigative Radiology, vol. 22, pp. 47-55, 1987.

Cheng et al., Cancer Res., 54:5547-5551, 1994.

Chiou et al., Org Lett., 2:347-350, 2000.

Cho and Dawson, J. Neurochem. 71:323-329, 1998.

Cho and Dawson, J. Neurochem., 74:xxxx., 2000.

Cho and Dawson, J. neurosci. Res., 60, 2000.

Cho et al., J. Neurosci. Res. 59: 32-38, 2000.

Choi et al., Cell, 53:519, 1988.

Chou and Fasman, Biochemistry, 13:211-222, 1974b.

Chou and Fasman, Ann. Rev. Biochem., 47:251-276, 1978b.

Chou and Fasman, Biophys. J., 26:367-384, 1979.

Chou and Fasman, Biochemistry, 13:222-245, 1974a.

Chou and Fasman, Adv. Enzymol. Relat. Areas Mol. Biol., 47:45-148, 1978a.

Cleary and Sklar, Proc. Nat'l. Acad. Sci. USA, 82:7439-43, 1985.

Cleary et al., J. Exp. Med., 164:315-20, 1986.

Cleary et al., Trends Microbiol., 4:131-136, 1994.

Cocea, Biotechniques, 23:814-816, 1997.

Coffer et al., Biochem. J. 335:1-13, 1998.

Cohen et al., Proc. Nat ′ Acad. Sci. USA 75:472-476, 1978.

Cook et al., “In vitro splicing of the ribosomal RNA precursor of Tetrahymena: involvement of a guanosine nucleotide in the excision of the intervening sequence,” Cell, 27:487-496, 1981.

Costa et al., Mol. Cell. Biol., 8:81, 1988.

Coupar et al., Gene, 68:1-10, 1988.

Crews et al., Proc. Natl. Acad. Sci. USA 93: 4316-4319, 1996.

Cripe et al., EMBO J., 6:3745, 1987.

Crowder and Freeman, J. Neurosci., 18:2933-2943, 1998.

Culotta and Hamer, Mol Cell. Biol., 9:1376, 1989.

Culver et al., Science, 256:1550-1552, 1992.

Dandolo et al, J. Virology, 47:55, 1983.

Das et al., J. Clin. Invest., 102:361-370, 1998.

Dawson et al., J. Neurochem., 68:2363-2370, 1997.

De Jager R, et al., “Current status of cancer immunodetection with radiolabeled human monoclonal antibodies” Semin Nucl Med 23(2):165-179, 1993.

De Villiers et al., Nature, 312:242, 1984.

Deamer and P. Uster, Liposomes (M. Ostro, ed.), Marcel Dekker, Inc., New York, pp. 27-52, 1983.

Deschamps et al., Science, 230:1174, 1985.

Dholakia et al., J. Biol. Chem., 264, 20638-20642, 1989.

Dong et al., Hum. Gene Ther., 7:319-331, 1996.

Doolittle MH and Ben-Zeev O,_“Immunodetection of lipoprotein lipase: antibody production, immunoprecipitation, and western blotting techniques” Methods Mol Biol., 109:215-237, 1999.

Dudler and Gelb, J. Biol. Chem., 271:11541-11547, 1996.

Duncan and Gilman, J. Biol. Chem. 273:15830-15837, 1998.

Duncan and Gilman, J. Biol. Chem. 271:23594-23600, 1996.

Dunphy et al., Biochim Biophys Acta.1436, 245-61, 1998.

Edbrooke et al., Mol. Cell. Biol., 9:1908, 1989.

Edlund et al., Science, 230:912, 1985.

El-Gorab et al., Biochem. Biophys. Acta, 1973, 306, 58-66, 1973.

Ellerby et al., Nature med. 5:1032-1038, 1999.

Fechheimer et al, Proc. Nat'l Acad. Sci. USA, 84:8463-8467, 1987.

Fendler et al., Catalysis in Micellar and Macromolecular Systems, Academic Press, New York, 1975.

Feng and Holland, Nature, 334:6178, 1988.

Fetrow and Bryant, Biotech., 11:479-483, 1993.

Firak and Subramanian, Mol. Cell. Biol., 6:3667, 1986.

Forster and Symons, Cell, 49:211-220, 1987.

Fraley et al., Proc. Nat ′ Acad. Sci. USA, 76:3348-3352, 1979.

Friedmann, Science, 244:1275-1281, 1989.

Frohman, PCR Protocols: A Guide To Methods And Applications, Academic Press, New York, 1990.

Fujita et al, Cell, 49:357, 1987.

Gregoriadis, ed., Drug Carriers In Biology And Medicine, pp. 287-341, 1979.

Gabizon et al., Cancer Res., 50:6371-8, 1990.

Gerlach et al., Nature (London), 328:802-805, 1987.

Ghose and Blair, Crit. Rev. Ther. Drug Carrier Syst., 3:263-359, 1987.

Ghosh and Bachhawat, In: Liver diseases, targeted diagnosis and therapy using specific receptors and ligands, (Wu G, Wu C ed.), New York: Marcel Dekker, pp. 87-104, 1991.

Gilles et al., Cell, 33:717, 1983.

Gloss et al., EMBO J., 6:3735, 1987.

Godbout et al., Mol. Cell. Biol., 8:1169, 1988.

Goodboum and Maniatis, Proc. Nat'l Acad. Sci. USA, 85:1447, 1988.

Goodboum et al., Cell, 45:601, 1986.

Gopal, Mol Cell Biol., 5:1188-1190, 1985.

Goswami and Dawson, J. Neurosci. Res., 60:141-9, 2000.

Goswami et al., J. Neurochem. 70:1376-1382, 1998.

Goswami et al., J. Neurosci. Res. 57:884-893, 1999.

Graham and Van Der Eb, Virology, 52:456-467, 1973.

Greene et al., Immunology Today, 10:272, 1989.

Gregoriadis, G., ed., Liposome Technology, vol. I, pp. 30-35, 51-65 and 79-107 (CRC Press Inc., Boca Raton, Fla., 1984.

Grosschedl and Baltimore, Cell, 41:885, 1985.

Gulbis B and Galand P,_“Immunodetection of the p21-Rasproducts in human normal and preneoplastic tissues and solid tumors: a review” Hum Pathol 24(12):1271-1285, 1993.

Gulbins et al., Immunity, 2:341-351, 1995.

Haimovitz-Friedman et al., Br. Med. Bull., 53:539-553, 1997.

Haklai and Kloog, Biochemistry: 37:1306-1314, 1998.

Han et al., J. Biol. Chem., 272:13432-13436, 1997.

Harlan and Weintraub, J. Cell Biol., 101: 1094-1099, 1985.

Harlow and Lane, Antibodies: A Laboratory Manual, Cold Spring Harbor Laboratory, 1988.

Haslinger and Karin, Proc. Nat 7Acad. Sci. USA., 82:8572, 1985.

Hauber and Cullen, J. Virology, 62:673, 1988.

Hellsten et al., EMBO J. 15:5240-5245, 1996.

Hen et al., Nature, 321:249, 1986.

Hensel et al., Lymphokine Res., 8:347, 1989.

Hermonat and Muzyczka, Proc. Nat'l Acad. Sci. USA, 81:6466-6470, 1984.

Herr and Clarke, Cell, 45:461, 1986.

Hirochika et al., J. Virol., 61:2599, 1987.

Hirsch et al., Mol. Cell. Biol., 10: 1959, 1990.

Hofinann et al., Neuropediatrics. 28:27-30, 1997.

Holbrook et al., Virology, 157:211, 1987.

Hollstein et al., Science 253:49-53, 1991.

Hope et al., Biochimica et Biophysica Acta, 812: 55-65, 1985.

Horlick and Benfield, Mol. Cell. Biol., 9:2396, 1989.

Horwich et al J. Virol., 64:642-650, 1990.

Huang et al., Cell, 27:245, 1981.

Hussussian et al., Nature Genetics, 15-21, 1994.

Huwiler et al., Biochimica Biophysica Acta., 1485:63-99, 2000.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Innis et al., Proc. Nat'l. Acad. Sci., 85:9436-40, 1988.

Inouye et al., Nucleic Acids Res., 13:3101-3109, 1985.

Irie & Morton, Proc. Nat'l Acad. Sci. USA 83:8694-8698, 1986.

Irie et al., In: Human Tumor Antigens and Specific Tumor Therapy, Metzgar & Mitchell (eds.), Alan R. Liss, Inc., New York, pp. 115-126, 1989.

Jacobson et al., Cell 88:347-54, 1997.

Jakobovits et al., Mol. Cell. Biol., 8:2555, 1988.

Jameel and Siddiqui, Mol. Cell. Biol., 6:710, 1986.

James and Olson, Biochem. 29:2623-2634, 1990.

Jameson and Wolf, Comput. Appl. Biosci., 4:181-186, 1988.

Jaynes et al., Mol. Cell. Biol., 8:62, 1988.

Johnson et al., J. Virol., 67:438-445, 1993.

Johnson et al., Mol. Cell. Biol., 9:3393, 1989.

Joyce, Nature, 338:217-244, 1989.

Kadesch and Berg, Mol. Cell. Biol., 6:2593, 1986.

Kaeppler et al., Plant Cell Reports 9: 415-418, 1990.

Kamb et al., Nature Genetics, 8:22-26, 1994.

Kamb et al., Science, 2674:436-440, 1994.

Kaneda et al., Science, 243:375-378, 1989.

Kaneda et al., J. Biol Chem., 264:12126-12129, 1989.

Kang et al., Science, 240:1034-1036, 1988.

Karin et al., Mol. Cell. Biol., 7:606, 1987.

Katinka et al., Cell, 20:393, 1980.

Katinka et al., Nature, 290:720, 1981.

Kato et al, J. Biol. Chem., 266:3361-3364, 1991.

Kawamoto et al., Mol. Cell. Biol., 8:267, 1988.

Kerr et al., Br. J. Cancer 26: 239-252, 1972.

Khatoon et al., Ann. of Neurology, 26, 210-219, 1989.

Kiledjian et al., Mol. Cell. Biol., 8:145, 1988.

Kim and Cech, Proc. Nat'l Acad. Sci. USA, 84:8788-8792, 1987.

King et al., J. Biol. Chem., 269, 10210-10218, 1989.

Klamut et al., Mol. Cell. Biol., 10:193, 1990.

Koch et al., Mol. Cell. Biol., 9:303, 1989.

Kohler et al., Methods Enzymol., 178:3, 1989.

Kraus et al. FEBS Lett., 428:165-170, 1998.

Kreier et al., Infection, Resistance and Immunity, Harper & Row, New York, (1991).

Kriegler and Botchan, In: Eukaryotic Viral Vectors, Y. Gluzman, ed., Cold Spring Harbor: Cold Spring Harbor Laboratory, N.Y., 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Krueger et al., J. Neuroscience. 15:3366-3374, 1995.

Kuhl et al., Cell, 50:1057, 1987.

Kunz et al., Nucl. Acids Res., 17:1121, 1989.

Kwoh et al., Proc. Nat. Acad. Sci. USA, 86: 1173, 1989.

Kyte and Doolittle, J. Mol. Biol., 157:105-132, 1982.

Labarca and Paigen, Anal. Biochem., 102:344-352, 1980.

Lareyre et al., J. Biol Chem., 274:8282-8290, 1999.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Lawrence et al., J. Med. Chem. 2: 4932-1941, 1999.

Lee et al., J. Auton Nerv Syst. 74:86-90, 1997.

Lee et al., Nature, 294:228, 1981.

Lenert et al., Science, 248:1639-1643, 1990.

Levenson et al., Hum Gene Ther. 20;9:1233-1236, 1998.

Levinson et al., Nature, 295:79, 1982.

Lin et al., Mol. Cell. Biol., 10:850, 1990.

Luria et al., EMBO J., 6:3307,1987.

Lusky and Botchan, Proc. Nat'l Acad. Sci. USA., 83:3609, 1986.

Lusky et al., Mol. Cell. Biol., 3:1108, 1983.

Macejak and Sarnow, Nature, 353:90-94, 1991.

Magee et al., EMBO J. 6:3353-3357, 1987.

Majors and Varmus, Proc. Nat'l Acad. Sci. USA., 80:5866, 1983.

Martin et al, Nature, 345:739-743, 1990.

Martinou et al., Neuron 13:1017-1030, 1994.

Mayer et al., Biochimica et Biophysica Acta, vol.858, pp.161-168, 1986.

Mayhew et al., Biochimica et Biophysica Acta, vol. 775, pp. 169-174, 1984.

Mayhew et al., Methods in Enzymology, vol. 149, pp. 64-77, 1987.

McNeall et al., Gene, 76:81, 1989.

Meng et al., Biochemistry 37: 10488-10492, 1998.

Michel and Westhof, J. Mol. Biol., 216:585-610, 1990.

Miksicek et al., Cell, 46:203, 1986.

Milligan et al., Tr. Biochem. Sci. 20:181-187, 1995.

Mitchell et al., Ann. N. Y Acad. Sci., 690:153-166, 1993.

Mitchell et al., J. Clin. Oncol,. 8:856-859, 1990.

Mizushima et al., FEBS Lett., 395:267-271, 1996.

Mordacq and Linzer, Genes and Dev., 3:760, 1989.

Moreau et al., Nucl. Acids Res., 9:6047, 1981.

Mori et al., Cancer Res., 54:3396-3397, 1994.

Morton D. L., and Ravindranath, In Tumor Immunology, Dalgleish AG (ed.), London: Cambridge University Press, 1-55, 1996.

Morton et al., Ann. Surg. 216: 463-482, 1992.

Muesing et al., Cell, 48:691, 1987.

Mumby, Curr. Opinion. Cell Biol. 9:148-154, 1997.

Nakagawa et al., Oncogene, 19:210-216, 2000.

Nakamura et al., In: Enzyme Immunoassays: Heterogeneous and Homogeneous Systems, Chapter 27, 1987.

Ng et al., Nuc. Acids Res., 17:601, 1989.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Nobri et al., Nature, 368:753-756, 1995.

Nomoto et al., Gene, 236:259-271, 1999.

O'Brien et al., J. Biol. Chem., 262:5210-5, 1987.

Obeid et al., Science, 259:1769-1771, 1993.

Ogilvie et al., J. Med. Chem. 40:4113-4135, 1997.

Ohara et al., Proc. Nat'l Acad. Sci. USA, 86: 5673-5677, 1989.

Okamoto et al., Proc. Nat ′ Acad. Sci. USA, 91:11045-11049, 1994.

Omirulleh et al., Plant Mol. Biol., 21:415-28, 1993.

Ondek et al., EMBO J., 6:1017, 1987.

Orlow et al., Cancer Res., 54:2848-2851, 1994.

Omitz et al., Mol Cell. Biol., 7:3466, 1987.

O'Shannessy et al., J. Immun. Meth., 99, 153-161, 1987.

Owens & Haley, J. Biol. Chem., 259:14843-14848, 1987.

Palmiter et al., Nature, 300:611, 1982.

Pech et al., Mol. Cell. Biol., 9:396, 1989.

Pelletier and Sonenberg, Nature, 334:320-325, 1988.

Perez-Stable and Constantini, Mol. Cell. Biol., 10: 1116, 1990. Physician's Desk Reference, (published: Montvale, N.J., Medical Economics).

Picard and Schaffner, Nature, 307:83, 1984.

Pinkert et al., Genes and Dev., 1:268, 1987.

Ponta et al., Proc. Nat ′ Acad. Sci. USA., 82:1020, 1985.

Porton et al., Mol. Cell. Biol., 10: 1076, 1990.

Potrykus et al., Mol. Gen. Genet., 199:183-188, 1985.

Potter & Haley, Meth. in Enzymol., 91, 613-633, 1983.

Queen and Baltimore, Cell, 35:741, 1983.

Quinn et al., Mol. Cell. Biol., 9:4713, 1989.

Ravindranath and Morton, Intern. Rev. Immunol. 7: 303-329, 1991.

Redondo et al., Science, 247:1225, 1990.

Reinhold-Hurek and Shub, Nature, 357:173-176, 1992.

Reisman and Rotter, Mol. Cell. Biol., 9:3571, 1989. Remington's Pharmaceutical Sciences, 15th Edition, Chapter 61, pages 1035-1038 and 1570-1580.

Resendez Jr. et al., Mol. Cell. Biol., 8:4579, 1988.

Ridgeway, In: Vectors: A survey of molecular cloning vectors and their uses, Rodriguez RL, Denhardt DT, ed., Stoneham:Butterworth, pp. 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Rosen et al., Cell, 41:813, 1988.

Rosenberg et al., Ann. Surg., 210:474, 1989.

Rosenberg et al., N. Engl. J. Med., 319:1676, 1988.

Sakai et al., Genes and Dev., 2:1144, 1988.

Salonen et al., Genome Res., 8:724-730, 1998.

Salvesen and Dixit Cell 91:443-446, 1997.

Sambrook et al., In: Molecular Cloning: A Laboratory Manual, 2nd Ed., Cold Spring Harbor Press, Cold Spring Harbor, N.Y., 1989.

Sarver, et al, Science, 247:1222-1225, 1990.

Sasso et al., J. Immunol., 142:2778-2783, 1989.

Satake et al., J. Virology, 62:970, 1988.

Scanlon et al., Proc. Nat'l Acad. Sci. USA, 88:10591-10595, 1991.

Schaffneretal., J. Mol. Biol., 201:81, 1988.

Schmidt et al., Biochim. Biophys. Acta 1257:205-213, 1995.

Schnolzer et al, Int. J. Pept. Prot. Res. 40:180-193, 1992.

Schroeder et al., J. Cell Biol. 134:647-660, 1996.

Searle et al., Mol Cell. Biol., 5:1480, 1985.

Sellers et al, J. Clin. Invest. 104: 1655-1661, 1999.

Serrano et al., Nature, 366:704-707, 1993.

Serrano et al., Science, 267:249-252, 1995.

Sharp and Marciniak, Cell, 59:229, 1989.

Shaul and Ben-Levy, EMBO J., 6:1913, 1987.

Sherman et al., Mol. Cell. Biol., 9:50, 1989.

Shimizu et al., Nature 374:811-816, 1995.

Shinoda, K. et al., Colloidal Surfactant, Academic Press, especially “The Formation of Micelles”, Ch. 1, 1-96, 1963.

Shorki et al., J. Immunol., 146:936-940, 1991.

Silvermann et al., J. Clin. Invest., 96:417-426, 1995.

Simmons et al., Biochemistry, 38:6346-6351, 1999.

Slee et al., J. Am. Chem. Soc. 117:11867-11878, 1995.

Sleigh and Lockett, J. EMBO, 4:3831, 1985.

Smith and Rutledge, Natl. Cancer Inst. Monogr., 42:141-143, 1975.

Soyombo and Hofinann, J. Biol. Chem. 272:27456-27463, 1997.

Spalholz et al., Cell, 42:183, 1985.

Spandidos and Wilkie, EMBO J., 2:1193, 1983.

Stephens and Hentschel, Biochem. J., 248:1, 1987.

Stuart et al., Nature, 317:828, 1985.

Sugimoto et al., J. Biol. Chem. 271:7705-11, 1996.

Sullivan and Peterlin, Mol. Cell. Biol., 7:3315, 1987.

Suopanki et al., Mol Genet Metab. 66:290-3, 1999b.

Suopanki et al., Neurosci Lett. 265:53-6, 1999a.

Swartzendruber and Lehman, J. Cell. Physiology, 85:179, 1975.

Szoka et al., Proc. Natl. Acad. Sci., 75:4194-4198, 1978.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Templeton et al., Nat. Biotechnol., 15:647-52, 1997.

Thiesen et al., J. Virology, 62:614, 1988.

Treisman, Cell, 42:889, 1985.

Tronche et al., Mol. Biol. Med., 7:173, 1990.

Trudel and Constantini, Genes and Dev., 6:954, 1987.

Tsujimoto et al., Science, 228:1440-3, 1985.

Tsujimoto, Croce, Proc. Natl. Acad. Sci. USA, 83:5214-8, 1986.

Tsumaki et al., J. Biol Chem. 273:22861-22864, 1998.

Tyndall et al., Nuc. Acids. Res., 9:6231, 1981.

Vannice and Levinson, J. Virology, 62:1305, 1988.

Vasseur et al., Proc. Nat'l Acad. Sci. USA., 77:1068, 1980.

Verheij et al., J. Biol. Chem 263:63-7, 1998.

Verkruyse and Hofmann, J. Biol. Chem. 271:15831-15836, 1996.

Vesa et al., Nature 376:584-587, 1995.

Vojtek and Der, J. Biol. Chem. 273:19925-19928, 1998.

Walker et al., Proc. Nat'l Acad. Sci. USA, 89:392-396 1992.

Wang and Calarne, Cell, 47:241, 1986.

Wang et al., Hepatology, 32:43-48, 2000.

Wawrzynczak & Thorpe, In: Immunoconjugates: Antibody Conjugates in Radioimaging and Therapy of Cancer, Vogel (ed.), New York, Oxford University Press, pp. 28-55, 1987.

Weber et al., Cell, 36:983, 1984.

Weinberg, Science, 254:1138-1145, 1991.

Weinberger et al Mol. Cell. Biol., 8:988, 1984.

Weinberger et al., Science, 228:740-742, 1985.

Wiesner and Dawson, Glycoconjugate J. 13:327-333, 1996b.

Wiesner and Dawson, J. Neurochem. 66:1418-1425, 1996a.

Winoto and Baltimore, Cell, 59:649, 1989.

Wolf et al., Comput. Appl. Biosci., 4:187-191, 1988.

Wong et al., Gene, 10:87-94, 1980.

Wu and Wu, J. Biol. Chem., 262:4429-4432, 1987.

Yang et al., Mol. Cell., 1:319-325, 1998.

Young et al., N. Engl. J. Med., 299:1261-1266, 1978.

Yutzey et al. Mol. Cell. Biol., 9:1397, 1989.

Zhang et al., Mol. Biotechnol., 8:223-231, 1997.

Zhao-Emonet et al., Gene Ther. 6:1638-1642, 1999.

Zhou et al., J. Biol. Chem., 273:16568-16575, 1998.

What is claimed is:

1. A method of inhibiting a cancer cell comprising administering to the cancer cell a composition comprising a PPT1 modulator in an amount effective to reduce PPTl activity level.

2. The method of claim 1, wherein inhibiting a cancer cell comprises altering proliferation, metastasis, contact inhibition, soft agar growth, cell cycle regulation, tumor formation, tumor progression, differentiation, programmed cell death, or tumor invasion.

3. The method of claim 1, wherein the PPT1 modulator comprises a proteinaceuous composition.

4. The method of claim 3, wherein the modulator competitively binds to PPT1.

5. The method of claim 4, wherein the modulator is an antagonist of PPT1.

6. The method of claim 1, wherein the modulator decreases the amount of PPT1.

7. The method of claim 1, wherein the modulator inhibits expression of PPT1.

8. The method of claim 4, wherein the modulator is at least one peptide or peptide mimetic that selectively interacts with PPT1.

9. The method of claim 8, wherein the modulator is at least one peptide that selectively interacts with PPT1.

10. The method of claim 8, wherein the modulator is at least one peptide mimetic that selectively interacts with PPT 1.

11. The method of claim 9, wherein the peptide comprises at least or at most 5 contiguous amino acids from SEQ ID NO:3.

12. The method of claim 11, wherein the peptide comprises the sequence VKIKK.

13. The method of claim 9, wherein the peptide comprises at least or at most 5 contiguous amino acids from SEQ ID NO:4.

14. The method of claim 13, wherein the peptide comprises the sequence YCWLR.

15. The method of claim 8, wherein the peptide or peptide mimetic is attached to a lipid component.

16. The method of claim 15, wherein the lipid component is a fatty acid.

17. The method of claim 16, wherein the fatty acid is unbranched.

18. The method of claim 15, wherein the lipid component is 8 to 30 carbons long.

19. The method of claim 18, wherein there is a double bond between C4 and C5.

20. The method of claim 15, wherein the peptide or peptide mimetic is attached to the lipid component through a non-hydrolyzable link.

21. The method of claim 15, wherein the lipid component comprises an oxime ether.

22. The method of claim 12, wherein the peptide is DAP1.

23. The method of claim 22, wherein DAP1 is in an α-ketoamide form.

24. The method of claim 8, wherein the modulator of PPT1 is a peptide mimetic.

25. The method of claim 24, wherein the modulator of PPT1 is a peptide mimetic of the amino acid sequence VKIKK.

26. The method of claim 24, wherein the modulator of PPT1 is a peptide mimetic of the amino acid sequence YCWLR.

27. The method of claim 24, wherein the peptide mimetic comprises a lipid component.

28. The method of claim 1, wherein the modulator of PPT1 is a nucleic acid containing a promoter operably linked to a PPT1 gene segment.

29. The method of claim 28, wherein the PPT1 gene segment is positioned, in reverse orientation, under the control of a promoter that directs expression of an antisense product.

30. The method of claim 28, wherein the nucleic acid encodes a ribozyme specific for an RNA transcript of PPT1 in a cell expressing an RNA transcript of PPT1.

31. The method of claim 4, wherein the modulator is an antibody composition comprising an antibody that recognizes PPT1.

32. The method of claim 1, further comprising administering to the cancer cell a composition comprising a chemotherapeutic drug.

33. The method of claim 1, wherein the cell is in a mammal.

34. A method of treating a subject with cancer comprising administering to the subject a PPT1 modulator in an amount effective to inhibit a cancer cell in the subject, thereby conferring a therapeutic benefit on the subject.

35. The method of claim 34, wherein the modulator is a peptide or peptide mimetic that selectively interacts with PPT1.

36. The method of claim 35, wherein the modulator is a peptide.

37. The method of claim 35, wherein the modulator is a peptide mimetic.

38. The method of claim 36, wherein the peptide comprises at least or at most 5 contiguous amino acids from SEQ ID NO:3.

39. The method of claim 38, wherein the peptide comprises the sequence VKIKK.

40. The method of claim 36, wherein the peptide comprises at least or at most 5 contiguous amino acids from SEQ ID NO:4.

41. The method of claim 40, wherein the peptide comprises the sequence YCWLR.

42. The method of claim 35, wherein the peptide or peptide mimetic is attached to a lipid component.

43. The method of claim 42, wherein the lipid component is a fatty acid.

44. The method of claim 39, wherein the peptide is DAP 1.

45. The method of claim 44, wherein DAP1 is in an α-ketoamide form.

46. The method of claim 34, further comprising treating the subject with a chemotherapeutic drug.

47. A method of screening a candidate substance for anti-cancer activity comprising:
(i) contacting a cancer cell with the candidate substance; and
(ii) assaying the compound's ability to modulate PPT1.

48. The method of claim 47, wherein modulation of PPT1 comprises altering PPT1 expression, activity, or location.

49. The method of claim 47, wherein assaying the compound's ability to modulate PPT1 comprises assaying for apoptosis.

50. The method of claim 49, further comprising administering a chemotherapeutic agent to the cell.

51. The method of claim 50, wherein the chemotherapeutic agent is administered to the cell prior to assaying for apoptosis.

52. The method of claim 47, wherein the cell is contacted in vitro.

53. The method of claim 47, wherein the cell is contacted in vivo.

54. A pharmaceutical composition comprising a recombinant vector containing an PPT1 gene segment positioned in reverse orientation, under the control of a promoter that directs expression of an antisense product.

55. A pharmaceutical composition comprising a peptide or peptide mimetic that selectively binds to PPT1 and that is covalently attached to a lipid component through a non-hydrolyzable linkage.

56. A pharmaceutical composition comprising a peptide mimetic that selectively binds to PPT1 and that is covalently attached to a lipid component through a non-hydrolyzable linkage.

57. A method of screening for cancer or pre-cancer in a subject comprising:
a) obtaining a sample from the subject;
b) assaying the sample for PPT 1 amount or activity level;
c) comparing the PPT1 amount or activity level of the subject to the PPT1 amount or activity level of a noncancerous sample, wherein elevated PPT1 amount or activity level may indicate cancer or pre-cancer in the subject.
